(19) United States
(12) Patent Application Publication
(10) Pub. No.: US 2023/0332238 A1
(43) Pub. Date: Oct. 19, 2023

(54) METHOD FOR CANCER SCREENING USING CAPILLARY BLOOD SAMPLE

(71) Applicant: SeekIn, Inc., Shenzhen (CN)

(72) Inventors: Guolin Zhong, Shenzhen (CN); Mao Mao, Shenzhen (CN); Yan Chen, Shenzhen (CN); Shiyong Li, Shenzhen (CN); Yumin Feng, Shenzhen (CN); Wei Wu, Shenzhen (CN)

(21) Appl. No.: 17/724,437

(22) Filed: Apr. 19, 2022

(51) Int. Cl. C12Q 1/6886; C12N 15/10; G01N 33/574

(52) U.S. Cl. CPC C12Q 1/6886; C12N 15/1065; C12N 15/1003; G01N 33/57484; C12Q 2600/118; C12Q 2600/156; C12Q 1/6869

(57) ABSTRACT

The disclosure relates to a method for separating circulating free nucleic acid (e.g., circulating free DNA, cfDNA) from capillary blood sample (e.g., capillary blood plasma) and methods of determining and/or assessing the risk of cancer in a subject.

### TECHNICAL FIELD

The present disclosure belongs to the biotechnology field, and in particular, the present disclosure relates to a method of isolating circulating free nucleic acids and constructing a sequencing library and its application thereof. More particularly, the present disclosure relates to a method of separating circulating free nucleic acid from capillary blood, constructing the library of the circulating free nucleic acid, and related sequencing and analyzing systems. The present disclosure also relates to a method of the detection of tumor markers using capillary blood sample.

### BACKGROUND

With the discovery of genes associated with cancer development, “liquid biopsy” is developed based on the change of the cancer-related gene mutation of ctDNA (circulating tumor DNA) from the patient's peripheral blood. Due to its high reproducibility, and the real-time monitoring of cancer dynamic changes, liquid biopsy has been widely recognized in clinical research. CtDNA is extracellular DNA that is present in the circulating plasma or serum, cerebrospinal fluid (CSF), and other bodily fluids of humans. It originates mainly from necrotic or apoptosis tumor cells, tumor cell secretion of outer discharge bodies and circulating tumor cells, and the peak size of ctDNA is usually 160-180 bp. As a special type of cfDNA (cell-free DNA), ctDNA can directly reflect the genetic information of patients with tumors. Compared to other conventional tumor markers, ctDNA has a short half-life, low false-positive rate, and their levels provide characteristics of the tumor in real-time. The next-generation sequencing of ctDNA can be used to detect and quantify cancer burden. In addition, previous reports show that ctDNA molecules are shorter than non-mutant cfDNA molecules and this feature can be utilized to improve the detection sensitivity. In patients with metastatic cancer, DNA fragments are found at higher concentrations than those in patients with localized cancers. Currently, conventional liquid biopsy techniques require at least 10 mL of venous peripheral blood to ensure sufficient cfDNA for detection. The collection of venous peripheral blood samples requires professional nurses to operate, which will bring certain limitations. In addition, the plasma separation from venous peripheral blood often requires centrifugation and transferring of the supernatant. Further removal of cell fragments and other impurities from the supernatant is also required. The centrifugation of large volume of liquid requires the use of large centrifuges, and the corner rotor that can be used on a high-speed centrifugal operation usually has only 6-8 channels. This flux greatly limits the clinical application, because of the long operational time.

Therefore, there exists a need for improved methods of detecting circulating tumor DNA. There is also a need for a cancer screening method based on next-generation sequencing with simple and convenient blood collection operation, low sample volume, and low-cost.

### SUMMARY

In one aspect, the disclosure provides a method of isolating circulating free nucleic acid from a capillary blood plasma sample, the method comprising:
(1) diluting no more than 500 μL of capillary blood plasma sample to a final volume of 1 mL using phosphate-buffered saline (PBS), thereby obtaining a diluted capillary blood plasma sample;
(2) lysing the diluted capillary blood plasma sample using protease K and buffer ACL, thereby obtaining a lysed capillary blood plasma sample;
(3) mixing the lysed capillary blood plasma sample with a binding buffer;
(4) loading the mixture in step (3) to a silica membrane column, to allow the binding of DNA to the silica membrane column;
(5) washing and drying the silica membrane column bound by DNA in step (4);
(6) eluting the DNA bound to the silica membrane column using 50-100 μL, preferably 50-60 μL of nuclease-free water (ddH₂O), thereby obtaining an eluted sample;
(7) loading the eluted sample to the silica membrane column, thereby obtaining the circulating free nucleic acid.

In some embodiments, the lysing comprises:
(a) mixing the protease K, a carrier RNA, a lysis buffer, and the diluted capillary blood plasma sample,
(b) incubating the mixture of (a) for about 20-40 minutes, preferably about 30 minutes at about 60° C., wherein the diluted capillary blood plasma sample has a volume of about 0.5-1.5 mL, preferably about 1 mL, the protease K has a volume of about 80-120 μL (preferably about 100 μL), the lysis buffer has a volume of about 750-850 μL (preferably about 800 μL), and the carrier RNA is used in an amount of about 0.5-1.5 μg (preferably about 1.0 μg).

In some embodiments, the mixture in step (3) is incubated on ice for about 5 minutes before step (4).

In some embodiments, the drying of step (5) is performed in a metal bath under about 56° C. for about 10 minutes.

In some embodiments, the plasma sample is obtained using the following steps:
(i) collecting no more than 500 μL of capillary blood using a tube containing K2-EDTA anticoagulant;
(ii) performing centrifugation on the capillary blood under 1600 g and about 4° C. for about 10 minutes, collecting the supernatant and removing the blood cells;
(iii) performing centrifugation on the supernatant obtained in step (ii) under 16000 g for 10 minutes, collecting the supernatant and removing impurities including cell debris, thereby obtaining the plasma.

In some embodiments, the method further comprises detecting and/or quantifying one or more protein tumor markers. In some embodiments, the protein tumor markers are selected from CEA, AFP, CA 125, CA 72-4, CA 19-9, CA 15-3, and CYFRA 21-1.

In another aspect, the disclosure provides a method for preparing a circulating free nucleic acid sequencing library from a capillary blood sample, the method comprising:
(a) end repairing and A-tailing the circulating free nucleic acid isolated by the method described herein, thereby obtaining A-tailed circulating free nucleic acid, wherein the starting amount of the circulating free nucleic acid is about 0.5-about 2.5 ng;
(b) ligating an adapter to the A-tail of the circulating nucleic acid, thereby obtaining an adapter-ligated library of circulating free nucleic acid;
(c) amplifying the adapter-tagged nucleic acid in the library.

In some embodiments, in step (b),

when the starting amount of the circulating free nucleic acid is not less than 0.5 ng and less than 1 ng, the concentration of the adapter used in the ligation is about 100-about 200 nM (preferably about 150 nM);

when the starting amount of the circulating free nucleic acid is not less than 1 ng and less than 2.5 ng, the concentration of the adapter used in the ligation is about 700-about 800 nM (preferably about 750 nM).

In some embodiments, in step (c), when the starting amount of the circulating free nucleic acid is not less than 0.5 ng and less than 1 ng, the amplification is performed for about 10-15 rounds (preferably about 12 rounds);
when the starting amount of the circulating free nucleic acid is not less than 1 ng and less than 2.5 ng, the amplification is performed for about 8-12 rounds (preferably about 10 rounds).

In some embodiments, in step (b), the concentration of adapter can be determined using the formula:

(CᵢVᵢ):(Mⱼ/(2LⱼMWⱼ))=about 100:1˜200:1 (e.g., about 162:1),

wherein Cᵢ is the molarity of the adapter, Vᵢ is the volume of the adapter, Mⱼ is the mass of the input cfDNA, Lⱼ is the length of the input cfDNA, and MWⱼ is the molecular weight of the dNTP.

In some embodiments, in step (c), the number of PCR cycles can be determined using the formula:

MᵢCRᵢARᵢPRᵢ₁PRᵢ₂(2ⁿ−2n)=Mⱼ

wherein Mᵢ is the mass of input DNA, CRᵢ is the library conversion efficiency, ARᵢ is the PCR amplification rate, PRᵢ₁ is the purification rate of the ligation products, PRᵢ₂ is the purification rate of the PCR products, n is the PCR cycle, and Mⱼ is the mass of the PCR product.

In another aspect, the disclosure provides a method of sequencing circulating free nucleic acid in a capillary blood sample, the method comprising: preparing a sequencing library using the method described herein; and sequencing the library thereby obtaining sequencing data.

In some embodiments, the circulating free nucleic acid is circulating free DNA (cfDNA).

In some embodiments, the capillary blood plasma sample is obtained from a subject having cancer, and the concentration of the isolated cfDNA is about 0.1-2 ng/μL.

In some embodiments, the method further comprises one or more purifying step(s) of the ligated library of circulating free nucleic acid to remove excess adapters.

In some embodiments, the purifying step(s) is performed using magnetic beads.

In some embodiments, the ratio of the volume of the magnetic beads and the volume of the ligated library of circulating free nucleic acid is about 2:1, about 1:1, or about 0.8:1 (preferably 0.8:1).

In some embodiments, method further comprises: analyzing the sequencing data to obtain information on genetic copy number variation and fragmentation pattern of the nucleic acid.

In another aspect, the disclosure provides a method of determining the probability of having cancer in a subject, the method comprising: obtaining no more than 500 μL of capillary blood plasma sample; diluting no more than 100 μL capillary blood plasma sample (preferably about 80 μL) 1 to 5 times (preferably 4 times); detecting protein tumor markers in the diluted plasma sample, wherein the protein tumor markers are one or more of CEA, AFP, CA 125, CA 72-4, CA 19-9, CA 15-3, and CYFRA 21-1; sequencing and analyzing circulating free nucleic acid in the remaining plasma sample using the method described herein, thereby obtaining information on gene copy number variation and fragmentation pattern; determining the probability of having cancer in the subject based on the gene copy number variation and the fragmentation pattern, and the detection of the protein tumor markers.

In another aspect, the disclosure provides device for sequencing the circulating free nucleic acid from capillary blood sample, the device comprising: a device for preparing a sequencing library for carrying out the method described herein; and a sequencing device for sequencing the library to obtain sequencing data.

In some embodiments, the device further comprises: a device for analyzing the sequencing data to obtain information on gene copy number variation and fragmentation pattern.

In another aspect, the disclosure provides a system for determining the probability of having cancer in a subject, the system comprising: a device to obtain no more than 500 μL of capillary blood plasma sample; a protein analyzing device, the device is designed to: dilute no more than 100 μL, preferably 80 μL of the plasma sample, thereby obtaining a diluted plasma sample, wherein the sample is diluted 1-5 times (preferably 4 times); and detect protein tumor markers in the diluted plasma sample, wherein the protein tumor markers are one or more of CEA, AFP, CA 125, CA 72-4, CA 19-9, CA 15-3, and CYFRA 21-1; a device for sequencing and analyzing circulating free nucleic acid in the rest of the capillary blood plasma sample, to obtain information on gene copy number variation and fragmentation pattern; a device for analyzing the results of the detection of the protein tumor markers, gene copy number variation and fragmentation pattern, thereby obtaining the probability of having cancer in a subject.

In another aspect, the disclosure provides a sequencing library, prepared by the method described herein.

In some embodiments, the circulating free nucleic acid is circulating free DNA (cfDNA).

In some embodiments, the capillary blood plasma sample is obtained from a subject having cancer, and cfDNA isolated from every 100 μL of blood sample is about 0.5-about 100 ng.

In some embodiments, the methods described herein further comprise one or more purifying step(s) of the ligated library of circulating free nucleic acid to remove excess adapters.

In some embodiments, the purifying step(s) is performed using magnetic beads.

In some embodiments, the ratio of the volume of the magnetic beads and the volume of the ligated library of circulating free nucleic acid is about 2:1 to about 0.8:1, preferably, the ration is about 0.8:1.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

FIG. 1 shows the result of incidence rate of blood coagulation in different brands of capillary blood collection tubes.

FIG. 2 shows the result of total comparison score of different brands of capillary blood collection tubes.

FIG. 3 shows a quality control merging figure for the cfDNA from 50 μL, 100 μL, 500 μL of T1 sample.

FIG. 4 shows the quality control figure of the sequencing library according to the method of the present disclosure.

FIGS. 5A-5F show the result of comparison of the CNV information of 50 μL plasma and 4000 μL plasma (shows above) and comparison of the CNV information of 100 μL plasma and 4000 μL plasma (shows below).

FIG. 6 shows the fragment size of cfDNA of 50 μL, 100 μL, and 4000 μL plasma by analyzing the sequencing data.

FIGS. 7A-7C show the result of comparison of the CNV information of the reaction system of 2 ng input DNA with ⅕ amount of library reagent and the reaction system of 44 ng input DNA with the amount of library reagent as usually.

FIG. 8 shows the fragment size of the reaction system of 2 ng input DNA with ⅕ amount of library reagent and the reaction system of 44 ng input DNA with the amount of library reagent as usually by analyzing the sequencing data.

FIGS. 9A-9C show the DNA distribution of library purified from 2×, 1× and 0.8× magnetic beads.

FIGS. 10A-10F show the DNA distribution of library from different input DNA with different adapter usage.

FIGS. 11A-11G show the comparison of the result of the tumor markers of the dilution plasma multiplying a dilute ratio and the result of the tumor markers of the non-dilution plasma.

### DETAILED DESCRIPTION

The disclosure provides a simple and convenient blood collection operation, less blood consumption, low-cost cancer screening method, to facilitate cancer screening method based on the next-generation sequencing to be more easily accepted by the public.

In one aspect, the disclosure provides a method of extracting/isolating circulating free nucleic acid from the capillary blood. The method includes the steps of (1) fixing the volume of the capillary blood plasma to 1 mL by adding PBS so as to obtain a plasma diluent; (2) carrying out lysis treatment on the plasma diluent by using protease K and buffer ACL (QIAGEN) so as to obtain lysis solution; (3) mixing the lysis solution with binding buffer solution; (4) loading a mixture obtained in the step (3) to a silica gel membrane column, and enabling the silica-gel membrane column to adsorb DNA; (5) sequentially cleaning and drying the silica-gel membrane column adsorbed with the DNA obtained in the step (4); (6) carrying out primary elution treatment on the silica gel membrane column adsorbed with the DNA by using nuclease-free water so as to obtain a primary eluent; and (7) carrying out secondary elution treatment on the silica gel membrane column subjected to primary elution by utilizing the primary eluent so as to obtain a solution containing the circulating free nucleic acid. Optionally, adjust 50 μL˜500 μL of plasma to 1 mL by adding PBS at step (1).

Also provided herein is a method of isolating circulating free nucleic acid from a capillary blood plasma sample, the method comprising:
(1) diluting no more than 500 μL of capillary blood plasma sample to a final volume of about 0.5-1.5 mL (preferably 1 mL) using phosphate-buffered saline (PBS), thereby obtaining a diluted capillary blood plasma sample;
(2) lysing the diluted capillary blood plasma sample using protease K and buffer ACL, thereby obtaining a lysed capillary blood plasma sample;
(3) mixing the lysed capillary blood plasma sample with a binding buffer;
(4) loading the mixture in step (3) to a silica membrane column, to allow the binding of DNA to the silica membrane column;
(5) washing and drying the silica membrane column bound by DNA in step (4);
(6) eluting the DNA bound to the silica membrane column using 50-100 μL, preferably 50-60 μL of nuclease-free water (ddH₂O), thereby obtaining an eluted sample;
(7) loading the eluted sample to the silica membrane column, thereby obtaining the circulating free nucleic acid.

In some embodiments, the washing and/or the eluting steps are repeated one or more times. In some embodiments, steps (5) and (6) are repeated one or more times using the loaded silica membrane column in step (4).

In some embodiments, the first elution treatment and the secondary elution treatment are performed independently by centrifugation at 200,000 g for about 1, 2, 3, 4, or 5 minutes. In some embodiments, the first elution treatment and the secondary elution treatment are performed independently by centrifugation at 200,000 g for about 1 minute. One of the advantages of the methods described herein is that sufficient nucleic acid samples can be obtained from relatively low levels of capillary blood. The sequencing results of circulating free DNA library indicate that the results obtained by the method are consistent with the test results of venous peripheral blood. So, the disclosure provides a simple and convenient blood collection operation, less blood consumption, low-cost cancer screening method, to facilitate cancer screening method based on the next-generation sequencing to be more easily accepted by the public.

In some embodiments, the lysis treatment further comprises: mixing protease K, carrier RNA, lysis buffer, and plasma dilution, incubating the mixture for about 10, 15, 20, 25, 30, 35, or more minutes at about 60° C. In some embodiments, the lysis treatment further comprises: mixing protease K, carrier RNA, lysis buffer, and plasma dilution, incubating the mixture for about 20, 25, 30, 35, or 40 minutes at about 60° C.

In some embodiments, the amount of protease K is about 50, 60, 70, 80, 90, 100, 110, 120, 130, 140 or 150 μl; the amount of lysis buffer is about 500, 600, 700, 800, 900, or 1000 μl; and the amount of carrier RNA is about 0.5, 0.6, 0.7, 0.8, 0.9, 1.0, 1.1, 1.2, 1.3, 1.4 or 1.5 μg for every 1 mL plasma diluent extraction.

In some embodiments, the lysis step (2) includes: (a) mixing the protease K, a carrier RNA, a lysis buffer, and the diluted capillary blood plasma sample, (b) incubating the mixture of (a) for about 15, 20, 25, 30, 35, 40, or 45 minutes at about 60° C., wherein the diluted capillary blood plasma sample has a volume of about 0.5-1 mL, the protease K has a volume of about 80-120 μL, the lysis buffer has a volume of about 750-850 μL, and the carrier RNA is used in an amount of about 0.5-1.5 μg.

In some embodiments, the amount of protease K is about 100 μl; the amount of lysis buffer is about 800 μl; and the amount of carrier RNA is about 1.0 μg for every 1 mL plasma diluent extraction.

In some embodiments, the mixture obtained in step (3) is pre-incubated on ice for about 1, 2, 3, 4, 5, 6, 7, 8, 9, or 10 minutes before performing step (4). In some embodiments, the mixture obtained in step (3) is pre-incubated on ice for about 5 minutes before performing step (4).

The drying treatment can be carried out in any suitable temperature. In some embodiments, in step (5), the drying treatment is carried out in a metal bath at about 56° C.

In some embodiments, the plasma is obtained following these steps: (i) using a K2-EDTA anticoagulant blood collection tube to collect about 50 μL to about 1000 μL of capillary blood sample. Preferably, 500 μL of capillary blood sample is obtained. Preferably, the collection tube is BD microtainer; (ii) The capillary blood is centrifuged at about 1,600 g of about 4° C. for about 5, 6, 7, 8, 9, 10, 11, 12, 13, 14, or 15 minutes, then transferring the supernatant. (iii) The supernatant obtained in step (ii) is centrifuged at about 16,000 g of about 4° C. for about 5, 6, 7, 8, 9, 10, 11, 12, 13, 14, or 15 minutes to further remove cell fragments and other impurities.

In some embodiments, the plasma is obtained following these steps: (i) using a K2-EDTA anticoagulant blood collection tube to collect about 50 μL to about 1000 μL of capillary blood sample. Preferably, 500 μL of capillary blood sample is obtained. Preferably, the collection tube is BD microtainer; (ii) The capillary blood is centrifuged at 1,600 g of 4° C. for about 10 minutes, then transferring the supernatant. (iii) The supernatant obtained in step (ii) is centrifuged at 16,000 g of 4° C. for about 10 minutes to further remove cell fragments and other impurities.

In some embodiments, the capillary blood plasma sample is obtained from a subject having cancer. In some embodiments, the capillary blood plasma sample is obtained from a subject that does not have cancer.

The volume of the blood sample in the methods described herein can be, for example, about 10 μL to about 5000 μL, about 50 μL to about 5000 μL, about 100 μL to about 5000 μL, about 500 μL to about 5000 μL, about 1000 μL to about 5000 μL, about 100 μL to about 3000 μL, about 500 μL to about 3000 μL, about 1000 μL to about 3000 μL, about 100 μL to about 1000 μL, about 100 μL to about 500 μL, about 200 μL to about 500 μL, about 300 μL to about 500 μL, or about 400 μL to about 500 μL.

In some embodiments, the volume of the capillary blood sample in the methods described herein is about 50 μL, about 100 μL, about 150 μL, about 200 μL, about 250 μL, about 300 μL, about 350 μL, about 400 μL, about 450 μL, about 500 μL, about 550 μL, about 600 μL, about 650 μL, about 700 μL, about 750 μL, about 850 μL, about 900 μL, about 950 μL, or about 1000 μL.

In some embodiments, about 5 μL, about 10 μL, about 15 μL, about 20 μL, about 25 μL, about 30 μL, about 35 μL, about 40 μL, about 45 μL, about 50 μL, about 100 μL, about 150 μL, about 200 μL, about 250 μL, about 300 μL, about 350 μL, about 400 μL, about 450 μL, about 500 μL, about 550 μL, about 600 μL, about 650 μL, about 700 μL, about 750 μL, about 850 μL, about 900 μL, about 950 μL, or about 1000 μL of plasma sample is obtained from the capillary blood sample.

The methods described herein provides a simple and effective way of isolating circulating free nucleic acid (e.g., cfDNA) from a relatively low volume of blood sample (capillary blood plasma sample). In some embodiments, the circulating free nucleic acid (e.g., cfDNA) isolated from every 100 μL of blood sample is about 0.5-100 ng, about 0.5-50 ng, about 0.5-10 ng, about 0.5-1 ng, about 1-100 ng, about 1-50 ng, about 1-10 ng, about 10-100 ng, or about 10-50 ng.

In another aspect, the disclosure provides a method of constructing sequencing library for cfDNA of capillary blood. In some embodiments, the method includes: (a) obtaining cfDNA according to the method described herein; (b) end repairing and A-tailing the cfDNA to obtain end-repaired, 5′phosphorylated, 3′-dA-tailed cfDNA fragments; (b) ligating adapters to the 3′-dA-tailed cfDNA fragments, thereby obtaining the ligation products with adapter; and (c) amplifying the ligation products to obtain sequencing library for cfDNA of capillary blood. In some embodiments, after step (b), the ligation product is purified. In some embodiments, after step (c), the sequencing library for cfDNA of capillary blood is purified.

In some embodiments, the purifying step(s) are carried out using magnetic beads. In some embodiments, the ratio of the volume of the magnetic beads and the volume of the ligated library of circulating free nucleic acid is about 0.8:1. In some embodiments, the purifying step(s) are carried out using magnetic beads. In some embodiments, the ratio of the volume of the magnetic beads and the volume of the ligated library of circulating free nucleic acid is about 1:1. In some embodiments, the purifying step(s) are carried out using magnetic beads. In some embodiments, the ratio of the volume of the magnetic beads and the volume of the ligated library of circulating free nucleic acid is about 2:1.

In some embodiments, the input of cfDNA in the methods described herein is about 0.5 to about 2.5 ng. In some embodiments, the input cfDNA is used to construct library and for sequencing.

In some embodiments, in step (b), the ligation reaction system contains about 10 μL, about 20 μL, about 30 μL, about 40 μL, or about 50 μL of ligation buffer; about 5 μL, about 10 μL, about 15 μL, or about 20 μL of DNA ligase; about 5 μL, about 10 μL, or about 15 μL of nuclease-free water; about 100 nM, about 150 nM, about 200 nM, about 250 nM, about 300 nM, about 350 nM, about 400 nM, about 450 nM, about 500 nM, about 550 nM, about 600 nM, about 650 nM, about 700 nM, about 750 nM, about 800 nM, about 850 nM or above of adapters (in a volume of about 5 μL, about 10 μL, or about 15 μL).

In some embodiments, in step (b), the ligation reaction system contains about 30 μL of ligation buffer, about 10 μL of DNA ligase, about 5 μL of nuclease-free water, about 5 μL of adapters (in a concentration of about 100-200 nM or about 700-800 nM, e.g., 150 nM for input DNA that is ≥0.5 ng and <1 ng; or 750 nM for input DNA that is ≥1 ng and ≤2.5 ng).

In some embodiments, when the input DNA is ≥0.5 ng and <1 ng, the concentration of the adapter used in the method described herein is about 100-200 nM. In some embodiments, when the input DNA is ≥1 ng and ≤2.5 ng, the concentration of the adapter used in the method described herein is about 700-800 nM.

In some embodiments, when the input DNA is ≥0.5 ng and <1 ng, the concentration of the adapter used in the method described herein is about 150 nM. In some embodiments, when the input DNA is ≥1 ng and ≤2.5 ng, the concentration of the adapter used in the method described herein is about 750 nM.

In some embodiments, the adapter-ligated cfDNA fragments are amplified. In some embodiments, the amplification is carried out using PCR. Any suitable number of cycles of the PCR reaction can be used in the methods described herein. For example, the number of PCR cycles in the amplification can be 5, 6, 7, 8, 9, 10, 11, 12, 13, 14, 15, 16, 17, 18, 19, 20, 21, 22, 23, 24, 25 or more. In some embodiments, in step (c), when the input DNA is ≥0.5 ng and <1 ng, the PCR cycle is set as 10-12; when the input DNA is ≥1 ng and 2.5 ng, the PCR cycle is set as 8-10. In some embodiments, in step (c), when the input DNA is ≥0.5 ng and <1 ng, the PCR cycle is set as 12; when the input DNA is ≥1 ng and ≤2.5 ng, the PCR cycle is set as 10.

In some embodiments, the blood sample used in the methods described herein is obtained from a subject having cancer. In some embodiments, the blood sample is a capillary blood sample.

In some embodiments, the cfDNA concentration obtained from the tumor blood sample correlates positively with the amount of blood sample (or the plasma sample obtained from the blood sample) used. In some embodiments, the existence of positive correlation indicates the presence of a tumor or cancer.

In some embodiments, the cfDNA isolated from every 100 μL of blood sample (e.g., the blood sample from the subject having tumor) is about 0.5-100 ng.

In some embodiments, the concentration of the cfDNA obtained from a subject can be used to determine, and/or assess the risk of cancer in a subject. In some embodiments, a subject is determined or predicted to have cancer if the cfDNA concentration obtained from the subject positively correlates with the amount of blood and/or plasma sample used to isolate the cfDNA.

In another aspect, the disclosure provides a method for sequencing the cfDNA of capillary blood. In some embodiments, the method comprises: constructing a sequencing library according to the method described herein; and sequencing the library in order to obtain sequencing data. In some embodiments, the library constructed is uses to sequence and analyze the sequencing data to assess the risk and probably of a subject having cancer.

In some embodiments, the method further comprises analyzing the sequencing data to analyze gene copy number variation and fragmentation patterns.

In another aspect, the disclosure provides a method of assessing the risk of cancer earlier based on cfDNA of capillary blood. In some embodiments, the method includes: acquiring no more than 500 μL plasma from the individual to be tested; adding a sample diluent to dilute no more than 100 μL plasma to obtain a plasma dilution, and the dilution ratio is about 1 to 5 fold; the plasma diluent is used to detect protein tumor markers. The tumor markers include one or more of CEA, AFP, CA 125, CA 72-4, CA 19-9, CA 15-3, and CYFRA 21-1;

In some embodiments, the methods provided herein further include sequencing and analyzing the mutation of cfDNA of capillary blood according to the method described herein to obtain the information of gene copy number variation and fragmentation patterns; assessing the cancer risk of the subjects based on the results of gene copy number variation, fragmentation patterns and protein tumor markers. The protein tumor marker used in the methods described herein are efficient and provide accurate test results for capillary blood samples such as fingerstick capillary blood.

In another aspect, the disclosure provides a device for sequencing the cfDNA of capillary blood. In some embodiments, the device comprises: a library construction system which is suitable for either of the preceding methods; a sequencing device which is suitable for the obtained libraries and is used to acquire sequencing data. Using this device, the method of sequencing cfDNA of capillary blood can be implemented effectively.

In some embodiments, the device further comprises: sequencing data analysis method for analyzing the sequencing data to obtain gene copy number variation and fragmentation patterns.

In another aspect, the disclosure provides a method of detecting cancer in a subject, assessing the risk of cancer in a subject, and/or determining the probability of a subject of having cancer by detecting tumor markers in a capillary blood sample. In some embodiments, the method includes: separating plasma from a capillary blood sample in the subject; diluting the plasma to obtain a diluted plasma sample, wherein the plasma is diluted about 1 to about 5 folds (preferably 4 folds); analyzing the diluted plasma sample to detect and/or quantify one or more tumor markers; and detecting and/or assessing the risk of cancer in the subject.

In some embodiments, the volume of the plasma sample is no more than 100 μL. In some embodiments, about 10 to about 100 μL of plasma sample is obtained from the subject. In some embodiments, about 80 μL of plasma sample is obtained from the subject.

The plasma sample is diluted before the detection and analysis of the tumor markers. In some embodiments, the plasma sample is diluted about 1, about 2, about 3, about 4, or about 5 folds. In some embodiments, the plasma sample is diluted about 4 folds.

In some embodiments, the final volume of the diluted plasma sample is no more than 300 μL. In some embodiments, the final volume of the diluted plasma sample is about 100 μL to about 300 μL. In some embodiments, about 80 μL of plasma sample is obtained from the subject and is diluted to a final volume of about 300 μL.

Any suitable tumor markers can be used for the detection and/or assessing of the risk of cancer described herein. In some embodiments, the protein tumor markers used in the methods described herein include CEA, AFP, CA 125, CA 72-4, CA 19-9, CA 15-3, and CYFRA 21-1.

In some embodiments, the detection and/or analysis of the tumor markers are performed using artificial intelligence (AI) methods.

In some embodiments, the methods described herein includes quantifying the level(s) of tumor markers (e.g., protein tumor markers) for the detection and/or assessing of the risk of cancer. Methods of quantifying tumor markers are known in the art.

In some embodiments, the methods described herein further includes sequencing and analyzing the mutation of cfDNA of capillary blood according to the method described above to obtain the information of gene copy number variation and fragmentation patterns. In some embodiments, the assessing of the risk of having cancer in the subject is based on the results of gene copy number variation, fragmentation patterns and the detection and analysis of the protein tumor markers.

Also provided herein are systems that can effectively carry out the methods described herein using capillary blood samples.

In another aspect, the disclosure provides a sequencing library that is constructed in accordance with the method described herein.

The methods an devices provided herein have certain advantages compared to other methods of isolating and analyzing circulating free nucleic acids. For example, the sample volume is lower than 500 μL, which allows the centrifugation to be carried out by a desktop high speed centrifuge. Compared to a large-volume high-speed centrifuge (usually 6-8 channels), the capacity of the desktop high speed centrifuge is greater and it save separation time. The capacity of the current methods and devices could reach 48 channels and the sample processing time can be as fast as about 10 minutes. This can prevent plasma from being degraded due to a long time at room temperature.

In some embodiments, about 10 to about 48 channels (each containing a separate plasma sample) are simultaneously processed. In some embodiments, the sample processing time is about 5, 6, 7, 8, 9, 10, 11, 12, 13, 14, 15, 16, 17, 18, 19, or 20 minutes. In some embodiments, the sample processing includes obtaining plasma from a blood sample (e.g., capillary blood sample).

In one aspect, the disclosure provides a method of detecting or assessing the risk of cancer in a subject, the method comprising one or more of the following steps: obtaining a capillary blood plasma sample in the subject; diluting the plasma to obtain a diluted plasma sample, wherein the plasma is diluted to more than 300 uL; analyzing the diluted plasma sample to detect and/or quantify one or more protein tumor markers; detecting and/or assessing the risk of cancer in the subject (e.g., using artificial intelligence). In some embodiments, the protein tumor markers are one or more of CEA, AFP, CA 125, CA 72-4, CA 19-9, CA 15-3, and CYFRA 21-1.

Additional aspects and advantages of the present invention will be given in part of the following description, and will become apparent from the following description, or known through the practice of the invention.

All numeric values in the disclosure are herein assumed to be modified by the term “about”, whether or not explicitly indicated. As used herein, the term “about” generally refers to a range of numbers that one of skill in the art would consider equivalent to the recited value (i.e., having the same function or result). In some embodiments, the terms “about” may include numbers that are rounded to the nearest significant figure. In some embodiments, the terms “about” may include numbers that are ±10%, ±20%, or ±30% of the value.

In the description of this specification, references to the terms “one embodiment”, “some embodiments”, “examples”, “concrete examples”, or “some examples”, etc. mean that the specific features, structures, materials, or features described in combination with such embodiments or examples are contained in at least one embodiment or example of the invention. In this specification, indicative representations of the above terms do not need refer to the same embodiments or examples. Furthermore, the specific features, structures, materials or features described may be combined in an appropriate manner in any one or more embodiments or examples. In addition, in the case of non-conflict, technicians in the field may combine together the different embodiments or examples described in this specification or the characteristics of the different embodiments or examples.

### EXAMPLES

The invention is further described in the following examples, which do not limit the scope of the invention described in the claims.

### Example 1: Methods

### Finger Stick Blood Collection

Required consumables: BD Microtainer (a disposable capillary blood collection container (Brand: BD, catalog number: 365974)), disposable blood collection device (Brand: BD, catalog number: 366594), alcohol pad.

The puncture site was found and an alcoholic pad was used to disinfect the puncture site. A disposable blood collection device was used. The disposable blood collection device was aimed at the puncture site and not removed before hearing the snap. A BD Microtainer was used and 500 μL of capillary blood was collected. The tube was inverted several times to mix the blood and avoid hemolysis by strenuous shaking.

### Plasma Separation

The equipment, reagents, and consumables needed for the experiment were prepared, and a high speed tabletop refrigerated centrifuge was pre-cooled to 4° C. in advance. The capillary blood was transferred from the collection tube to 500 μL centrifuge tube. The parameters were set to be: temperature at 4° C., centrifugal force of 1600 g, time for 10 min. After balancing the centrifuge tube, it was placed in a centrifuge for centrifugation. After the centrifugation was completed, the centrifuge tube was placed on a centrifuge tube rack in biological safety cabin. The supernatant was transferred into a new 500 μL centrifuge tube, and marked with the sample number and operating time on the tube wall. The supernatant should be carefully collected to avoid sucking in white blood cells. The parameters were set as: temperature at 4° C., centrifugal force of 16,000×g, and time for 10 min. The 500 μL centrifuge tube containing the supernatant was balanced and placed into a centrifuge for centrifugation. After the centrifugation was completed, the centrifuge tube containing the supernatant was placed on a centrifuge tube rack in the biological safety cabin. Transfer the supernatant into a new 5 mL tube. The supernatant should be carefully collected to avoid sucking in the precipitate. The purpose of this step was to remove impurities such as cell debris in the plasma. The plasma and blood cells were placed in a refrigerator at −80° C. for later use.

Circulating-Free DNA (cfDNA) Extraction

The equipment, reagents, and consumables required for the experiment were prepared. A water bath was switched on and adjusted to the temperature of 60° C. A heating block was switched on and adjusted to the temperature of 56° C. Extraction was performed using the QIAamp Circulating Nucleic Acid Kit (Brand: Qiagen, catalog number: 55114), prepare buffers and reagents (Buffer ACB, Buffer ACW1, Buffer ACW2, ACL mixture and dissolve carrier RNA to Buffer ACL) per the manufacturer's instructions.

Phosphate-buffered saline was added into plasma to a final volume of 1 mL. 100 μL proteinase K was pipetted into the above centrifuge tube, and vortexed intermittently for 30 s. 0.8 mL Buffer ACL (containing 1.0 μg carrier RNA) was added. The cap was closed and mixed by pulse-vortexing for 30 s. Make sure that a visible vortex forms in the tube. To ensure efficient lysis, it was essential that the sample and Buffer ACL were mixed thoroughly to yield a homogeneous solution. Note: Do not interrupt the procedure at this time. Lysis incubation was performed immediately after and the sample was incubated at 60° C. for 30 min. 1.8 mL Buffer ACB was added to the lysate in the tube. The cap was closed and mixed thoroughly by pulse-vortexing for 15 s. The lysate-Buffer ACB mixture was incubated in the tube for 5 min on ice or in refrigerator.

### Assembling of the Suction Filtration Device:

The QIAvac 24 Plus was connected to a vacuum source. A VacValve was inserted into each luer slot of the QIAvac 24 Plus. A VacConnector was inserted into each VacValve. The QIAamp Mini columns were inserted into the VacConnectors on the manifold. Finally, a tube extender (20 mL) was inserted into each QIAamp Mini column. Make sure that the tube extender was firmly inserted into the QIAamp Mini column to avoid leakage of sample. Note: the 2 mL collection tube was remained for the subsequent operation. The sample number was marked on the QIAamp Mini silica membrane column. VacValve ensured a steady flow rate. VacConnectors prevented direct contact between the spin column and VacValve during purification, thereby avoiding any cross-contamination between samples. The QIAamp Mini silica membrane column adsorbs DNA, and the tube extender can hold large volumes of plasma. The lysate-Buffer ACB mixture was carefully applied into the tube extender of the QIAamp Mini column. The vacuum pump was switched on. When all lysates have been drawn through the columns completely, the vacuum pump was switched off and the exhaust valve was opened to release the pressure to 0 mbar. The tube extender was carefully removes and discarded. 600 μL Buffer ACW1 was applied to the QIAamp Mini column. The exhaust valve was closed and the vacuum pump was switched on. After all of Buffer ACW1 have been drawn through the QIAamp Mini column, the vacuum pump was switched off and the exhaust valve was opened to release the pressure to 0 mbar.

750 μL Buffer ACW2 was applied to the QIAamp Mini column. The exhaust valve was closed and the vacuum pump was switched on. After all of Buffer ACW2 have been drawn through the QIAamp Mini column, the vacuum pump was switched off and the exhaust valve was opened to release the pressure to 0 mbar. 750 μL ethanol (96-100%) was applied to the QIAamp Mini column. The exhaust valve was closed and the vacuum pump was switched on. After all of the ethanol have been drawn through the QIAamp Mini column, the vacuum pump was switched off and the exhaust valve was opened to release the pressure to 0 mbar. The lid of the QIAamp Mini column was closed and removed from the vacuum manifold. The VacConnector was discarded. Place the QIAamp Mini column in a clean 2 mL collection tube, and centrifuged at full speed (20,000×g; 14,000 rpm) for 3 min. The QIAamp Mini Column was placed into a new 2 mL collection tube. The lid was opened, and the assembly was incubated at 56° C. for 10 min to dry the membrane completely. The QIAamp Mini column was placed in a clean 1.5 mL elution tube (included in the kit), and the 2 mL collection tube was discarded. 55 μL of nuclease-free water was carefully applied to the center of the QIAamp Mini membrane. The lid was closed and incubated at room temperature for 3 min and centrifuged in a microcentrifuge at full speed (20,000×g; 14,000 rpm) for 1 min to elute the nucleic acids. The elution buffer from above steps was carefully applied to the center of the QIAamp Mini membrane. The lid was closed and incubated at room temperature for 3 min and centrifuged in a microcentrifuge at full speed (20,000×g; 14,000 rpm) for 1 min to elute the nucleic acids.

### CfDNA Library Construction

Preparation Before the Library Construction:

The magnetic beads were taken out of the refrigerator at 4° C. and incubated at room temperature for 30 minutes before use. End Repair & A-Tailing Buffer and End Repair reagent & A-Tailing Buffer enzyme mix were taken out of the refrigerator at −20° C. and thawed on the ice box. The details about the name, sampling date, and DNA concentration were recorded on the experimental record books and each sample numbered. Some 200 μL PCR tubes were taken and marked with numbers (both the cap and the wall of the tube were labeled). The KAPA Hyper Prep Kit (Roche, Cat. No. kk8504) was used for the sequencing library construction.

### End Repair & A-Tailing

The end repair & A-Tailing reaction system was prepared according to Table 1.

TABLE 1
End repair & A-Tailing reaction systems

| | | 8 reaction systems | |
|---|---|---|---|
| Component | 1 reaction system | (excess 5%) | |
| End Repair & | 7 μL | 58.8 | μL |
| A-Tailing Buffer | | | |
| End Repair & | 3 μL | 25.2 | μL |
| A-Tailing enzyme mix | | | |
| Total volume | 10 μL | 84 | μL |

10 μL of the above-mentioned end repair reaction system was added to each 200 μL PCR tube with 50 μL cfDNA from above steps, mixed well, and centrifuged at low speed. The thermocycler was set to perform the program as shown in Table 2.

TABLE 2
End Repair and A-Tailing

| Step | Temperature | Time |
|---|---|---|
| End Repair and A-Tailing | 20° C. | 30 min |
| | 65° C. | 30 min |
| HOLD | 4° C. | ∞ |

The reaction system was taken out of the thermocycler and placed on the small yellow plate, and carried out an adapter ligation reaction.

Adapter Ligation Reaction System:

The adapter ligation reaction system was prepared according to Table 3.

TABLE 3
The adapter ligation reaction system

| | | 8 reaction systems |
|---|---|---|
| Component | 1 reaction system | (excess 5%) |
| Ligation Buffer | 30 μL | 252 μL |
| DNA Ligase | 10 μL | 84 μL |
| Nuclease-Free water | 5 μL | 42 μL |
| Total volume | 45 μL | 378 μL |

40 μL of the above reaction system was added to each reaction tube, mixed gently, and centrifuged at low speed.

An appropriate amount of adapter corresponding to the amount of input DNA was added. Adapter and insert molar ratio were as shown in Table 4. 5 μL of the adapter was added to each reaction tube. In addition, according to the sequencing requirements, each sample was added with a unique adapter, to avoid the situation that two samples using the same adapter occurred on the same lane. The information about the adapters used in each sample was well recorded.

TABLE 4
Adapter and insert molar ratio

| The amount of input DNA | Adapter concentration |
|---|---|
| 1 ng ≤ X ≤ 2.5 ng | 750 nM |
| 0.5 ng ≤ X < 1 ng | 150 nM |

The above reaction system was mixed well and placed into the PCR amplifier, the temperature was set to be 20° C., and reacted for 15 min.

### DNA Purification

80% ethanol (for example, 50 mL of 80% ethanol: 40 mL of absolute ethanol+10 mL of nuclease-free water) was prepared before use.

The corresponding number of 1.5 mL sample tubes was prepared and marked.

The magnetic beads, which have been pre-equilibrated at room temperature, were fully vortexed and mixed, 88 μL of which was added into each tube.

The above DNA mixture was mixed with the magnetic beads, and incubated at room temperature for 10 min.

The 1.5 mL tube was placed on the magnet to capture the magnetic beads until the liquid became clear.

The supernatant was carefully removed and discarded, then 200 μL of 80% ethanol was added into the tube. The tube was rotated 360 degrees horizontally and incubated on the magnet at room temperature for 30 s, and then the supernatant was discarded. (During this process, the centrifuge tube had been kept on the magnet.)

The above steps were repeated once.

The residual ethanol was removed without disturbing the beads. The cap of the tube was open to dry the magnetic beads at room temperature and volatilized the ethanol, preventing the effect of the enzyme in the subsequent reaction system from being affected by the excess ethanol. Note: the magnetic beads should not be excessively dried, otherwise the DNA will not be easily eluted from the magnetic beads, resulting in reduced yield. The drying should be stopped once the surface of the magnetic beads is no longer shiny.

21 μL of nuclease-free water was added into each sample tube to resuspend the magnetic beads. They were mixed well and incubated at room temperature for 5 min.

A new batch of 200 μL PCR tubes was prepared and marked with the corresponding sample number on the wall and cap of the tube.

The tube was placed on the magnet to capture the magnetic beads until the solution was clear, then the supernatant was transferred to the corresponding PCR tube as a template for the PCR experiment.

### Library Amplification

The library amplification reaction system was prepared according to Table 5.

TABLE 5
The library amplification reaction system

| | | 8 reaction systems |
|---|---|---|
| Component | 1 reaction system | (excess 5%) |
| 2 × KAPA HiFi Hotstart | 25 μL | 210 μL |
| Ready Mix | | |
| 10 × KAPA Library | 5 μL | 42 μL |
| Amplification Primer mix | | |
| Total master mix volume | 30 μL | 252 μL |

30 μL of Pre-PCR amplification reaction system was added to each 200 μL PCR tube containing cfDNA-adapter products, mixed gently and centrifuged at low speed, and then placed in the thermocycler for reaction.

The thermocycler was set as the following program, and the PCR cycles were be adjusted appropriately according to the amount of input DNA, as shown in Table 6.

TABLE 6
PCR reaction system

| | | Reaction | | |
|---|---|---|---|---|
| Step | Temperature | time | | Cycle number |
| Preliminary | 98° C. | 45 | s | 1 |
| denaturation | | | | |
| Denaturation | 98° C. | 15 | s | Refer to the cycle |
| Annealing | 60° C. | 30 | s | number selection reference |
| Elongation | 72° C. | 30 | s | table for specific cycle |
| | | | | number |
| Final elongation | 72° C. | 1 | min | 1 |

| | | | |
|---|---|---|---|
| Storage | 4° C. | ∞ | 1 |

The selection of cycle number was shown in Table 7.

TABLE 7
Selection of cycle number

| The amount of input DNA | PCR cycles |
|---|---|
| 1 ng ≤ X ≤ 2.5 ng | 10 |
| 0.5 ng ≤ X < 1 ng | 12 |

After the Pre-PCR reaction was finished, the library purification began.

### DNA Library Purification

The corresponding number of 1.5 mL sample tubes was prepared and marked.

The magnetic beads, which have been pre-equilibrated at room temperature, were fully vortexed and mixed, 50 μL of which was added into each tube.

The above DNA mixture was mixed with the magnetic beads, and incubated at room temperature for 10 min.

The 1.5 mL tube was placed on the magnet to capture the magnetic beads until the liquid become clear.

The supernatant was carefully removed and discarded, then 200 μL of 80% ethanol was added into the tube. The tube was rotated 360 degrees horizontally and incubates on the magnet at room temperature for 30 s, and then the supernatant was discarded. (During this process, the centrifuge tube had been kept on the magnet.)

The above steps were repeated once.

All residual ethanol was removed without disturbing the beads. The cap of the tube was open to dry the magnetic beads at room temperature and volatilize the ethanol, preventing the effect of the enzyme in the subsequent reaction system from being affected by the excess ethanol. Note: the magnetic beads should not be excessively dried, otherwise the DNA will not be easily eluted from the magnetic beads, resulting in reduced yield. The drying should be stopped once the surface of the magnetic beads is no longer shiny.

35 μL of nuclease-free water was added into each sample tube to resuspend the magnetic beads, mixed well and incubated at room temperature for 5 min.

A new batch of 200 μL PCR tubes was prepared and marked with the corresponding sample number on the wall and cap of the tube.

The tube was placed on the magnet to capture the magnetic beads until the solution was clear, then the supernatant was transferred to the corresponding PCR tube as a template for the PCR experiment.

1 μL of cfDNA library was taken for quantitative determination and insert DNA size was detection using Agilent 2100 bioanalyzer. Record the information.

The samples were placed in the freezer boxes of the corresponding item and stored at −20° C.

### Library Pooling and Sequencing

The equipment, reagents, and consumables needed for the experiment were prepared. A pooling volume of each sample was calculated according to the concentration of library and the sequence depth. A new 1.5 mL centrifuge tube was taken and labeled. Each sample was subjected to pooling in the same 1.5 mL centrifuge tube according to the calculated volume. Ensure the adapter of the samples were unique in a pool. After mixing thoroughly to yield a homogeneous solution, the concentration was measured, and the information was recorded. The above pooled library was diluted and denatured with Tris-HCl and NaOH, and then sequenced on an Illumina sequencing system with 2×150 bp for WGS.

### Analysis of Sequencing Data

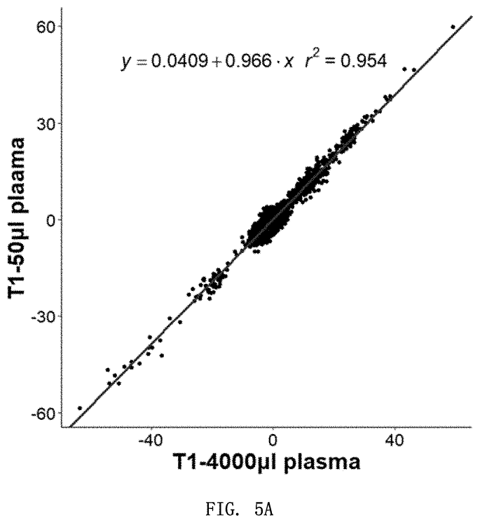

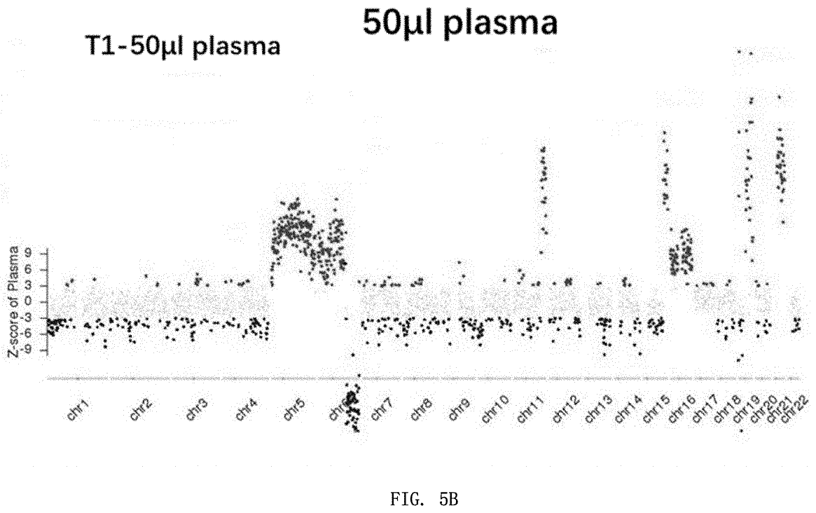

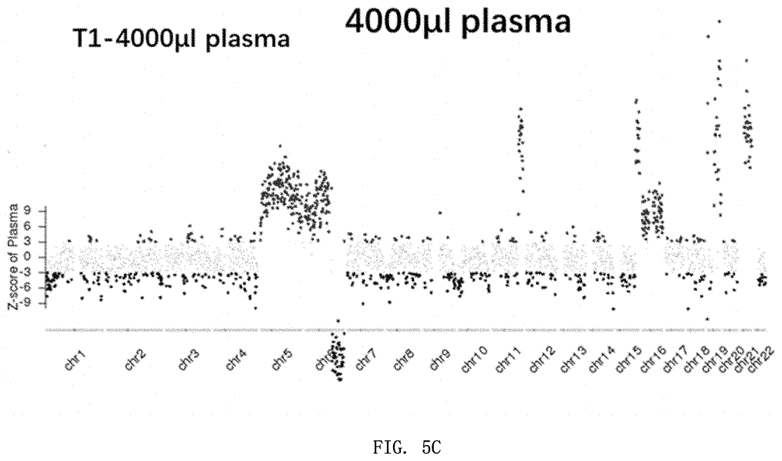

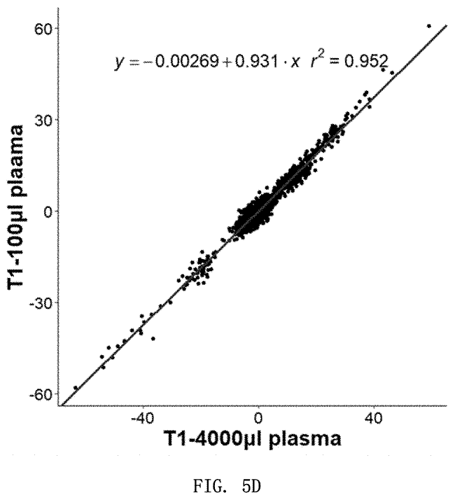

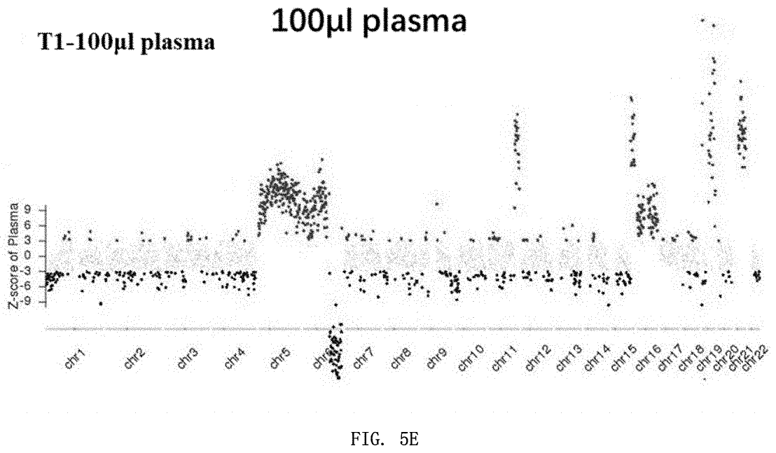

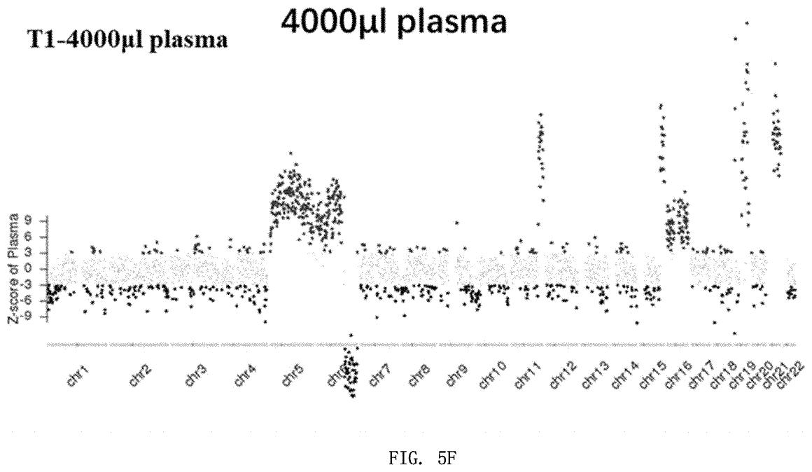

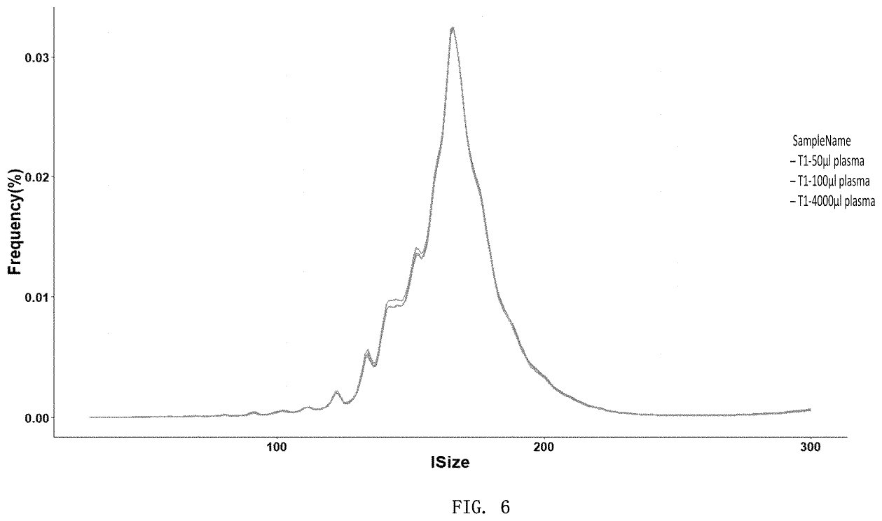

(1) According to the method of the above embodiment, sequencing data was obtained. After filtering out low-quality reads, an alignment software (BWA) was used to align these sequencing reads to the human reference genome (hg19).
(2) The mapping results were filtered, a mapping quality score was required to be greater than 30, and duplicate reads as well as reads that were not proper pair alignment, etc., were removed. The reference genome was divided into bins, each of the bins was 10,000 bp, and the comparison reads of each bin were counted.
(3) The filtering of bins includes: 1) mappability >0.5; 2) a ratio of N<0.5; 3) not in the region files wgEncodeDacMapabilityConsensusExcludable.bed and wgEncodeDukeMapabilityRegionsExcludable.bed downloaded from UCSC; 4) filtering out X and Y chromosomes; 5) filtering out bins with large standard deviation of all bins;
(4) According to GC ratio of each bin: the number of A, T, C, and G bases in each window (bin), and the number of G and C were counted. Mappability calculation: according to the ENCODE's mappability bigwig file downloaded from UCSC, the mappability of each region in the file was compared with the bin, and an average mappability of all regions in each bin was calculated as the mappability value of the bin.
(5) The GC ratio and mappability of each bin were combined, the bins were grouped according to the combination thereof, and a median number of reads of all bins corresponding to each combination of GC and mappability.
(6) Using a generalized cross-validation method, the bins were divided into 10 parts on average, most parts (such as 9) of which were used to fit non-parametric regression curve by locally weighted scatterplot smoothing (LOESS), and the remaining 1 part was used as the test set to predict, calculate AIC. The optimal value (AIC minimum) of locally weighted nonparametric regression parameters was determined. The fitted curves were constructed and finally the corrected values were obtained by dividing the standardised depths of each bins by the values predicted by the curves.
(7) In the healthy cohort, the corrected depths at the same bin satisfy the normal distribution. Therefore, calculate the mean and standard deviation (SD) of the normal distribution of each bin based on the healthy cohort. Z-score of each bins was calculated by subtracting the mean value and dividing it by SD value. If the absolute value of the subject's Z-score is greater than 3, it is considered that this bin of the sample was deletion or amplification in this region, as show in FIGS. 5A-5F.
(8) Based on the results of the alignment, pairs of reads participating in the genome in normal comparison were selected, and the length of the inserted fragment was calculated according to the start and end locations of pairs of reads. The number of reads corresponding to different lengths of insert fragments was counted, and the length distribution of insert fragments was shown in FIG. 6.

### Detection of Protein Tumor Markers

80 μL plasma obtained from the fingerstick capillary blood sample was decanted for detection of protein tumor markers. The protein tumor markers include CEA, AFP, CA 125, CA 72-4, CA 19-9, CA 15-3, and CYFRA 21-1. The Cobas e411 (Roche Diagnostics GmbH, Mannheim, Germany) was utilized to measure the concentration of these seven protein tumor markers with the test reagents supporting the platform.

The routine maintenance, calibration and quality control of the instruments were carried out regularly before sample testing.

Then add 240 μL dilution buffer into 80 μL plasma for a 4-fold dilution. And the mixture was decanted for analyze these seven protein tumor markers.

The protein quantification before dilution was obtained by multiplying the detection result by dilution factor 4.

### Example 2: Capillary Blood Collection Container Selection

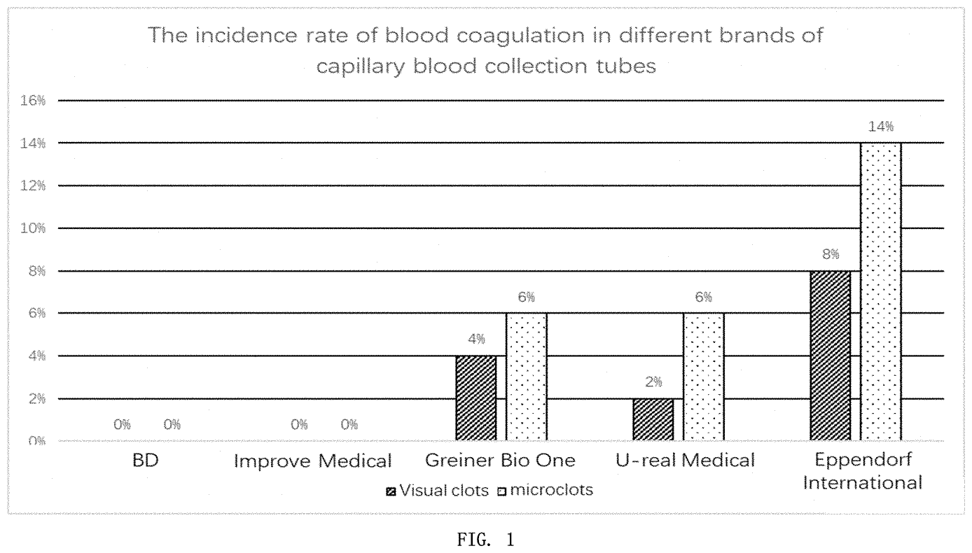

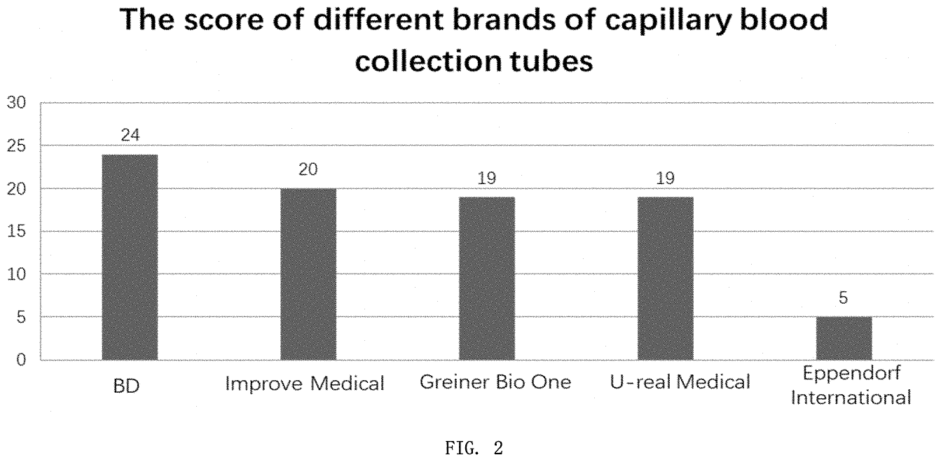

The method described herein includes exploring a variety of capillary blood collection containers, including: Microtainer (Brand: BD), Vaccutee MiniColect (Brand: Greiner Bio One), Impromini (Brand: Improve Medical), GD005 (Brand: U-Real Medical), Safe Lock Tubes (Brand: Eppendorf International). FIG. 1 shows the result of incidence rate of blood coagulation in different brands of capillary blood collection tubes. FIG. 2 shows the result of total comparison score of different brands of capillary blood collection tubes. The rating contents include: appearance, easy to open and close the cap, easy to collect, inner wall smoothness, and sealing. Among them, the BD Microtainer (a disposable capillary blood collection container) was preferred. Among them, the BD Microtainer has three features: (1) The bionic inner wall can accelerate sampling rate, avoid blood cell attachment wall to induce hemolysis, mix does not in time and induce interfere with detection results. (2) Simultaneously safety head cover double concave design and PET material tubes prevent accidental injuries when opening the head cover or transportation. (3) Preset high concentration K2-EDTA anticoagulant accurately ensure sample quality. As a result, BD Microtainer (a disposable capillary blood collection container) (Brand: BD, catalog number: 365974) was selected and disposable blood collection device (Brand: BD, catalog number: 366594) for capillary blood collection was selected. After many blood collection tests, it was found that the blood collection process was convenient and simple. It is easy to operate at home without professional training. And it is not easy to generate hemolysis and coagulation.

### Example 3: Plasma Separation from Capillary Blood

In the method described herein, the total blood volume was lowered to about 500 μL, compared to other traditional methods, so that centrifugation could be carried out by a desktop high speed centrifuge, and the capacity could reach 48 channels. Compared with a large-volume high-speed centrifuge (usually 6-8 channels), the capacity of the desktop high speed centrifuge is greater and it save separation time. This can prevent plasma from being degraded due to a long time at room temperature. The processing time for plasma separation (e.g., for at least 64 samples) can be about or less than 10 minutes.

### Example 4. Extraction of Low-volume Plasma and Library Construction

The method described herein used a normal sample and a cancer sample and separates plasma from capillary blood, respectively. 50 μL, 100 μL, and 500 μL capillary blood sample were used for cfDNA extraction, respectively, and then used to construct libraries, and the cfDNA concentration is shown in Table 8 below.

TABLE 8
cfDNA concentration
cfDNA concentration (ng/μL)

| Sample | Plasma volume | | |
|---|---|---|---|
| No. | 50 μL | 100 μL | 500 μL |
| T1 | 0.232 | 0.34 | 1.24 |
| N1 | 0.168 | 0.14 | 0.162 |

Surprisingly, the results of the method described herein shows that in T1 (tumor samples), the cfDNA concentration was positively correlated with plasma, but this relationship was not present in N1 (normal human sample), which may be due to adding Carrier RNA during our extraction process. The Carrier RNA was a kind of Poly-A, which was 0.5-4 kb, mainly to promote the cfDNA binding to the filter column membrane and reduce some non-specific binding during extraction. This carrier RNA still existed in the extracted cfDNA, and would also be quantified by the Qubit fluorescent. In normal people, the cfDNA content was very low, so the concentration detected by Qubit was not correlated with the amount of plasma. And because Carrier RNA was a single-strand, the adapter ligation reaction could not be performed during the subsequent library construction. So, the residual Carrier RNA will not influence the library construction and the sequencing data analysis. At the same time, the final elution was carried out twice during the cfDNA extraction process, and 55 μL of Nuclease-Free water was added to the filtration column, and after centrifugation, the eluate was added to the filtration column for centrifugation again. The purpose of 2 times of elution was to increase the elution efficiency and reduce cfDNA residues on the filter column membrane. The filtration column was QIAamp Mini column which was used in the steps of cfDNA extraction. The binding buffer prompted the cfDNA to bind to the membrane, and some impurities were removed using different concentrations of buffer and ethanol. Finally, the DNA was eluted from the membrane with water.

As can be seen from the following results, the cfDNA concentration of the 2 times of elution was significantly higher than 1 time elution. 23% higher than the overall average, and the details are shown Table 9 below.

TABLE 9
Elution systems
Comparison of the cfDNA concentration of elution of one time and two times

| | First elution | | Second elution | | |
|---|---|---|---|---|---|
| Sample | concentration | Volume-1 | concentration | Volume-2 | Percentage |
| No. | (ng/μL) | (μL) | (ng/μL) | (μL) | increase |
| S1 | 0.87 | 54 | 1.11 | 53 | 25% |
| S2 | 0.354 | 54 | 0.414 | 53 | 15% |
| S3 | 0.55 | 54 | 0.65 | 53 | 16% |
| S4 | 4.14 | 54 | 5.74 | 53 | 36% |
| S5 | 0.244 | 54 | 0.356 | 53 | 43% |
| S6 | 0.43 | 54 | 0.494 | 53 | 13% |
| S7 | 0.55 | 54 | 0.622 | 53 | 11% |
| S8 | 0.31 | 54 | 0.414 | 53 | 31% |
| S9 | 0.338 | 54 | 0.448 | 53 | 30% |
| S10 | 0.696 | 54 | 0.834 | 53 | 18% |
| S11 | 0.652 | 54 | 0.77 | 53 | 16% |

| | |
|---|---|
| Average | 23% |

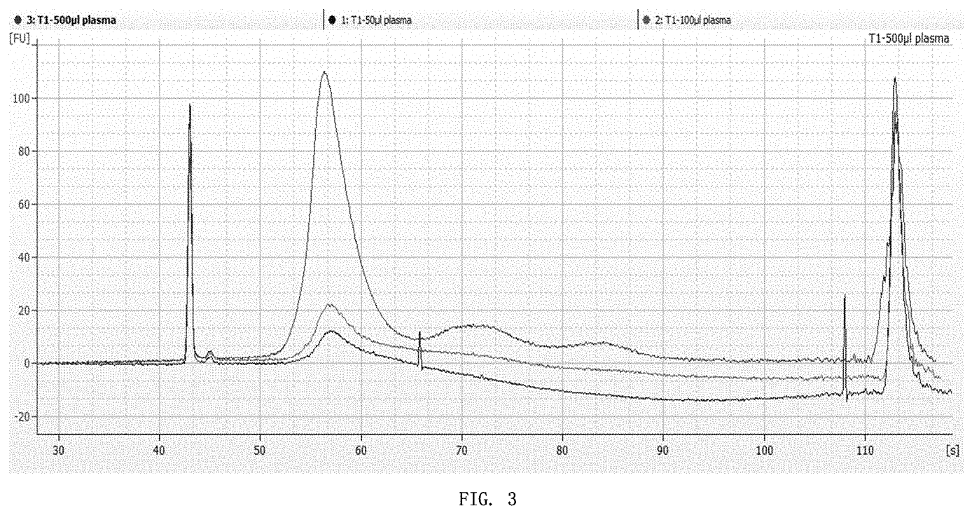

These cfDNA fragments were further tested for fragment size distribution. FIG. 3 shows that a quality control merging figure for the cfDNA from 50 μL, 100 μL, 500 μL of T1 sample. It can be seen that the position of the cfDNA obtained by different plasma amounts is consistent, and there is no degradation and genomic DNA (gDNA) contamination. Then construct the cfDNA library, and the details are shown in Table 10 below.

TABLE 10
Library comparison

| | | cfDNA | Input | adapter | | Library | Library |
|---|---|---|---|---|---|---|---|
| Serial | | concentration | cfDNA | concentration | PCR | concentration | volume |
| No. | Sample No. | (ng/μL) | (ng) | (μM) | cycle | (ng/μL) | (μL) |
| 1 | T1-50 μL | 0.232 | 11.6 | 3 | 7 | 12.8 | 35 |
| | plasma | | | | | | |
| 2 | T1-100 μL | 0.34 | 17 | 7.5 | 7 | 24.4 | 35 |
| | plasma | | | | | | |
| 3 | T1-500 μL | 1.24 | 31 | 7.5 | 6 | 26 | 35 |
| | plasma | | | | | | |
| 4 | N1-50 μL | 0.168 | 8.4 | 3 | 8 | 1.12 | 35 |
| | plasma | | | | | | |
| 5 | N1-100 μL | 0.14 | 7 | 3 | 8 | 1.81 | 35 |
| | plasma | | | | | | |
| 6 | N1-500 μL | 0.162 | 8.1 | 3 | 8 | 7.44 | 35 |
| | plasma | | | | | | |

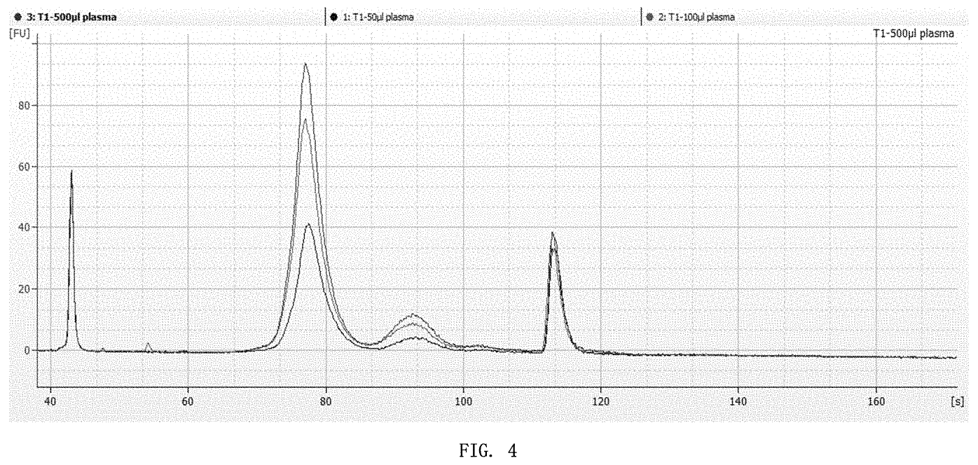

FIG. 4 shows the quality control figure of the sequencing library according to the method described herein. It can be seen that the fragment size distribution of cfDNA library fragments extracted from different plasma amounts is basically consistent. The libraries of the above samples are sequenced by Novaseq 6000, and analyze the sequencing data.

### Example 5: Comparison of Sequencing Data

First, gene copy number variation (CNV) of the low plasma amount was analyzed and the result compared with the result of cfDNA from 4000 μL plasma. FIGS. 5A-5C shows a result of comparison the CNV information of 50 μL plasma and 4000 μL plasma and a result of comparison the CNV information of 100 μL plasma and 4000 μL plasma. It can be seen that the gene CNV of 50 μL plasma or 100 μL plasma has a good consistency with the gene CNV of 4000 μL plasma. Its correlation coefficient R² is at least 0.952 (0.954 for 50 μL plasma VS 4000 μL plasma, and 0.952 for 100 μL plasma VS 4000 μL plasma).

Next, the fragment size distribution of the low plasma was analyzed, and the result compared with the fragment size distribution of cfDNA from 4000 μL plasma. FIG. 6 shows the fragment size of cfDNA of 50 μL, 100 μL, and 4000 μL plasma by analyzing the sequencing data. It can be seen that the fragment size distribution of 50 μL plasma or 100 μL plasma has a good consistency with the fragment size distribution of 4000 μL plasma. In summary, the current disclosure provides that even in the case of 50 μL (extremely low plasma quantity), the CNV variation of and the fragment size distribution of cfDNA can be detected sensitively by the methods described herein.

### Example 6: Input DNA and the Amount of Reagents for Library Construction

Due to the decrease of plasma, the amount of cfDNA will decrease. In order to prove the same detection effect can be achieved by reducing the amount of reagent in the library, we have done a series of experiments. The details are shown in Table 11:

TABLE 11
Library construction systems

| | | cfDNA | Sampling | Water | Input | | Index | | Library |
|---|---|---|---|---|---|---|---|---|---|
| Serial | Sample | concentration | volume | volume | cfDNA | The amount | concentration | PCR | concentration |
| No. | No. | (ng/μL) | (μL) | (μL) | (ng) | of Reagent | (μM) | cycle | (ng/μL) |
| 1 | T2 | 2.2 | 20 | 30 | 44 | 1 | 7.5 μM | 5 | 19.9 |
| 2 | T2 | 2.2 | 0.91 | 9.09 | 2 | 1/5 | 750 nM | 10 | 20.2 |

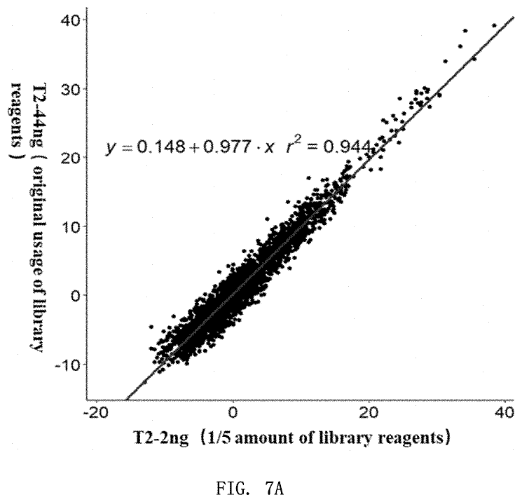

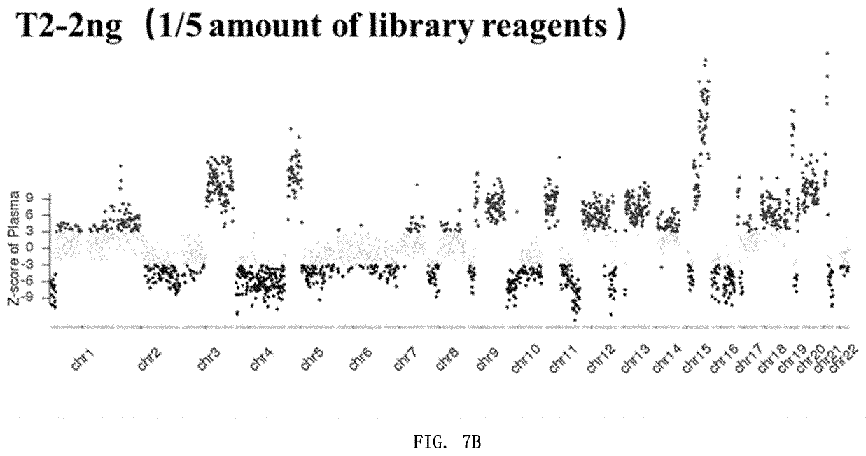

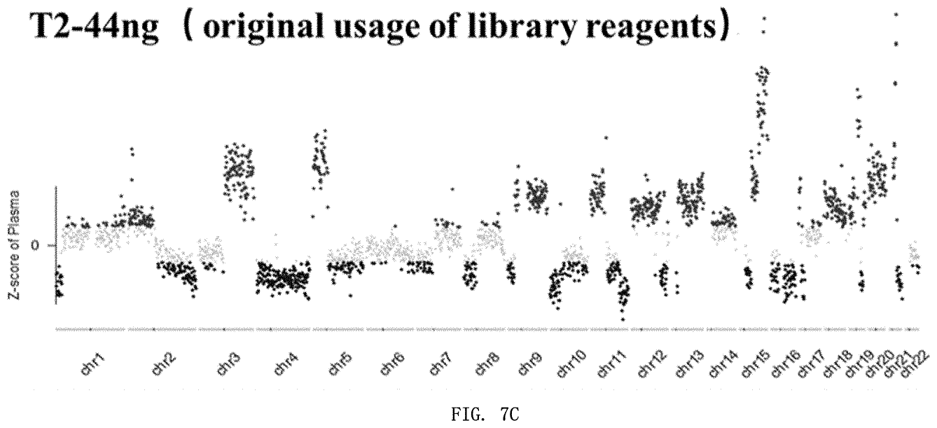

First, gene CNV was analyzed. FIGS. 7A-7C show a result of comparison the CNV information of the reaction system of 2 ng input DNA with ⅕ amount of library reagent and the reaction system of 44 ng input DNA with the amount of library reagent as usually. It can be seen that the gene CNV of the reaction system of 2 ng input DNA with ⅕ amount of library reagent has a good consistency with the reaction system of 44 ng input DNA with the amount of library reagent. Its correlation coefficient R² is 0.944.

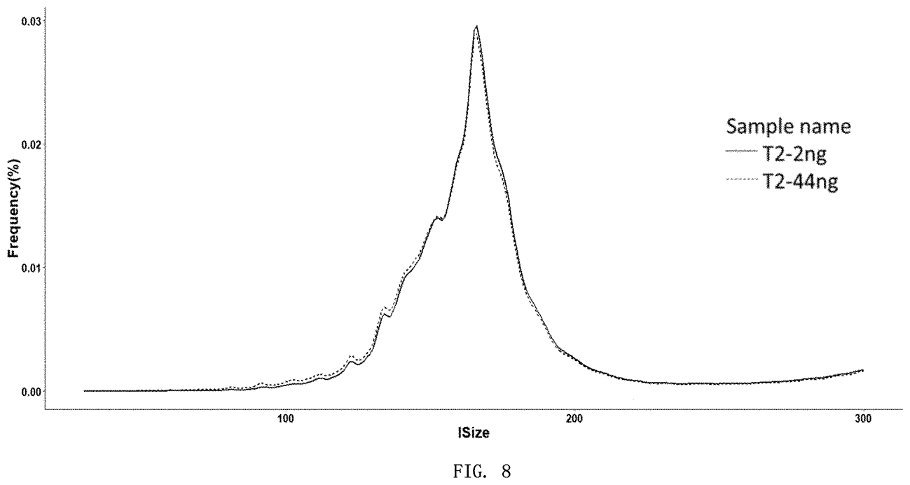

Next, the fragment size distribution of was analyzed. FIG. 8 shows the fragment size of the reaction system of 2 ng input DNA with ⅕ amount of library reagent and the reaction system of 44 ng input DNA with the amount of library reagent as usually by analyzing the sequencing data. It can be seen that the fragment size distribution of the reaction system of 2 ng input DNA with ⅕ amount of library reagent has a good consistency with the fragment size distribution of the reaction system of 44 ng input DNA with the amount of library reagent as usually. Even the amount of library building reagent is reduced to ⅕, the CNV variation and fragment size distribution information of ctDNA can be detected by using the methods described herein.

### Example 7: Optimization of Library Construction

The amount of adapter in the ligation reaction, the amount of magnetic beads in the library purification step after ligation reaction, and the PCR cycle number in PCR amplification reaction were optimized.

(1) The Amount of Magnetic Beads in the Purification Step after the Ligation Reaction:

Generally, slightly excessive adapter is added in the process of ligation reaction to improve the efficiency of ligation reaction, which causes some adapter dimer remaining in the ligation reaction. So it needs to be removed by adjusting the amount of magnetic beads in the subsequent purification step. The principle of magnetic beads separating DNA is that the buffer of suspended magnetic beads contains PEG and salt ions which drive DNA can be adsorbed to the surface of carboxyl modified polymer magnetic beads. This process is reversible. Under appropriate conditions, the bound DNA molecule can be eluted. DNA of different fragment sizes can be adsorbed to magnetic beads by adjusting the amount of magnetic beads and buffer, thus achieving the purpose of DNA sorting. And the longer DNA fragment preferentially be absorbed on the beads. As the amount of beads increasing, the smaller the DNA fragment is adsorbed to the magnetic beads.

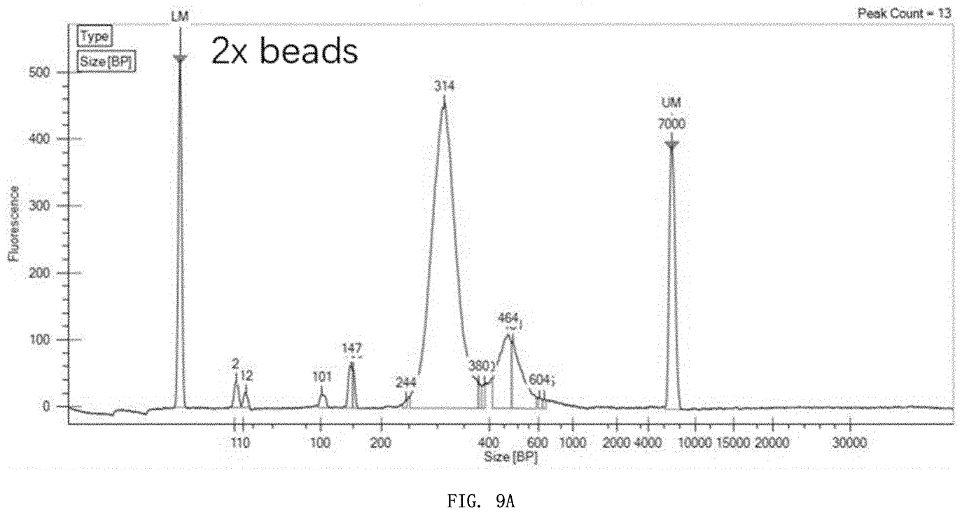

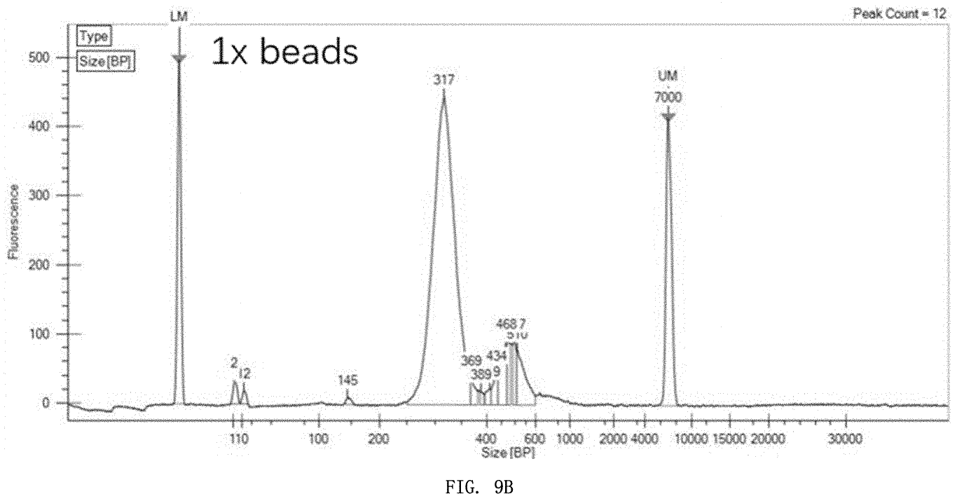

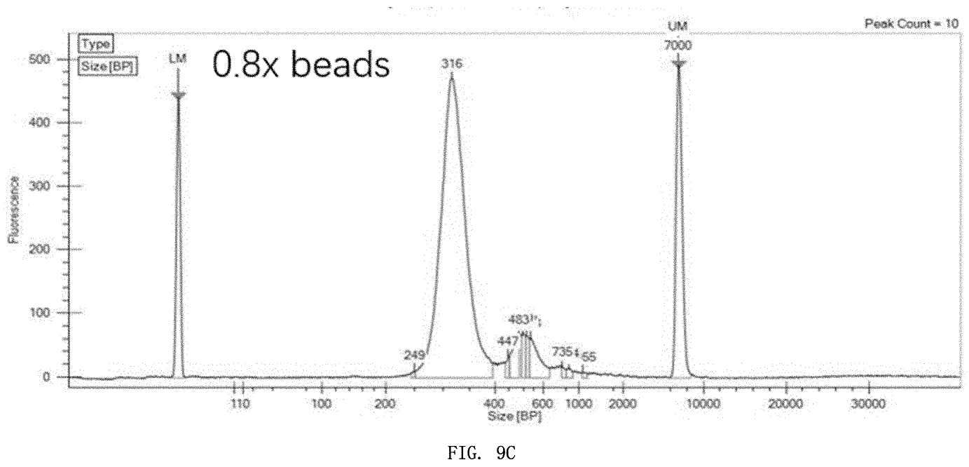

In the current experiment, 2×, 1× and 0.8× magnetic beads (v/v, compared to the volume of the cfDNA-containing sample, e.g. ligated-cfDNA sample) were used for subsequent purification steps. For example, the reaction system of the adapter ligation including ligated-cfDNA was 110 and 110 μL of magnetic beads were added in, and this is called the 1× group. The size of the purified fragments was detected by LabChip. According to the analysis of adapter dimer residues in the purified products, it can be seen from FIGS. 9A-9C that there are obvious adapter dimers in the libraries with the dosage of magnetic beads 2× and 1×, and adapter dimers are more obvious in the former library than in the latter. Therefore, the optimal amount of magnetic beads in the magnetic bead purification step after the adapter ligation reaction is 0.8× (e.g., 88 μL of magnetic beads added to 110 μL of ligated cfDNA reaction).

(2) Optimization of the Amount of Adapter in the Ligation Reaction and the Number of PCR Cycles in PCR Amplification:

TABLE 12
Library construction parameter tests

| | Input | Index | | Library | Library |
|---|---|---|---|---|---|
| Sample | cfDNA | concentration | PCR | concentration | volume |
| No. | (ng) | (μM) | cycle | (ng/μL) | (μL) |
| S12 | 0.5 | 150 nM | 16 | 55 | 35 |
| S13 | 0.5 | 150 nM | 16 | 64 | 35 |
| S14 | 0.5 | 150 nM | 14 | 30.4 | 35 |
| S15 | 0.5 | 150 nM | 14 | 39.6 | 35 |
| S16 | 0.5 | 150 nM | 14 | 40.4 | 35 |
| S17 | 0.5 | 150 nM | 14 | 29 | 35 |
| S18 | 0.5 | 150 nM | 14 | 22.6 | 35 |
| S19 | 0.5 | 150 nM | 14 | 31 | 35 |
| S20 | 0.5 | 150 nM | 14 | 40.2 | 35 |
| S21 | 1 | 100 nM | 13 | 39.6 | 35 |
| S22 | 1 | 100 nM | 13 | 41.2 | 35 |
| S23 | 1 | 100 nM | 13 | 42.8 | 35 |
| S24 | 1 | 100 nM | 13 | 45.6 | 35 |
| S25 | 2 | 750 nM | 14 | 77.2 | 35 |
| S26 | 2 | 750 nM | 14 | 68.6 | 35 |
| S27 | 2 | 750 nM | 10 | 10.3 | 35 |
| S28 | 2 | 750 nM | 10 | 10.2 | 35 |
| S29 | 2 | 750 nM | 10 | 12.8 | 35 |
| S30 | 2 | 750 nM | 10 | 20.4 | 35 |
| S31 | 2 | 750 nM | 10 | 20.2 | 35 |
| S32 | 2 | 750 nM | 10 | 14.6 | 35 |

Through the above tests, it was found that the adapter concentration and the PCR cycles should be adjusted to prevent adapter residual for high concentration of adapter, and to prevent a high duplication rate for overmuch PCR cycles. That will influence the sequencing output and data.

TABLE 13
Library construction parameter optimization

| | Input | | | Library |
|---|---|---|---|---|
| Sample | cfDNA | Adapter | PCR | concentration |
| No. | (ng) | concentration | cycles | (ng/ul) |
| S33 | 0.5 | 750 nM | 14 | 35 |
| | 0.5 | 375 nM | 14 | 29.4 |
| | 0.5 | 150 nM | 14 | 30.7 |
| S34 | 2.5 | 1.5 μM | 12 | 38.5 |
| | 2.5 | 750 nM | 12 | 32.1 |
| | 2.5 | 375 nM | 12 | 17.8 |

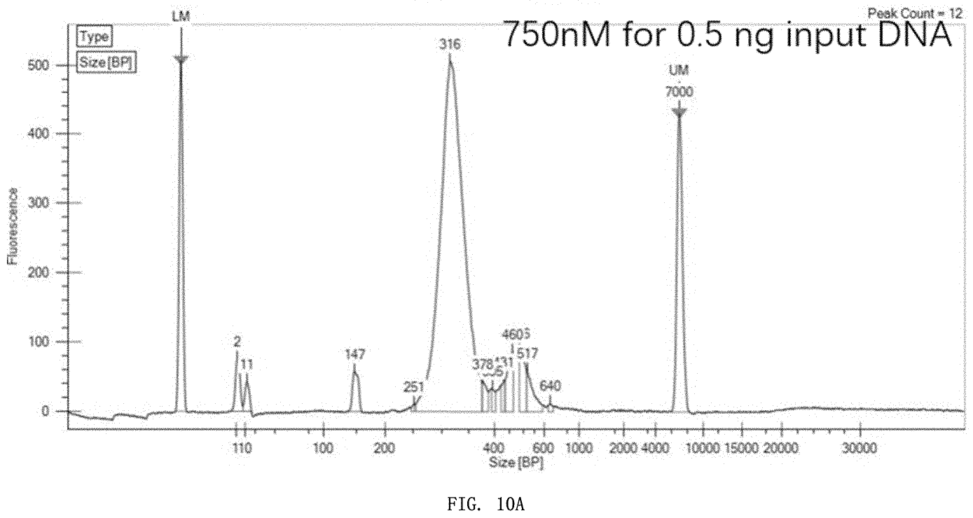

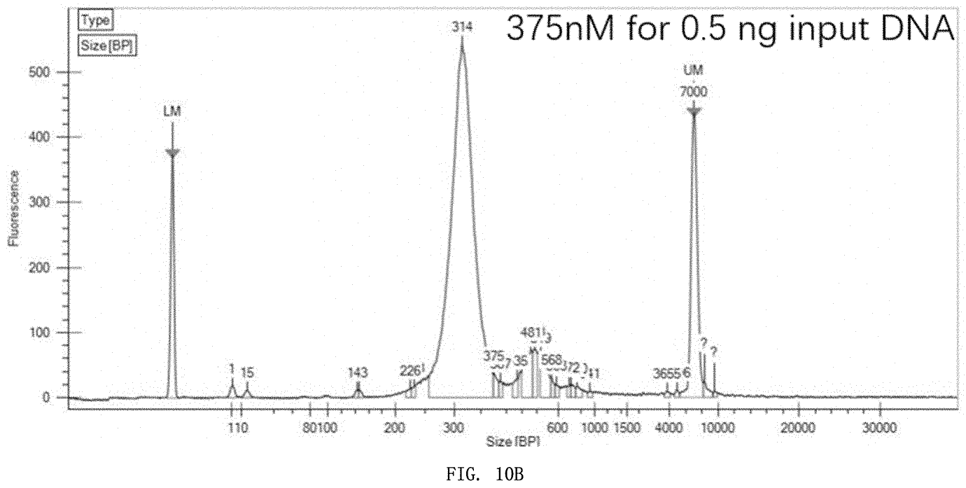

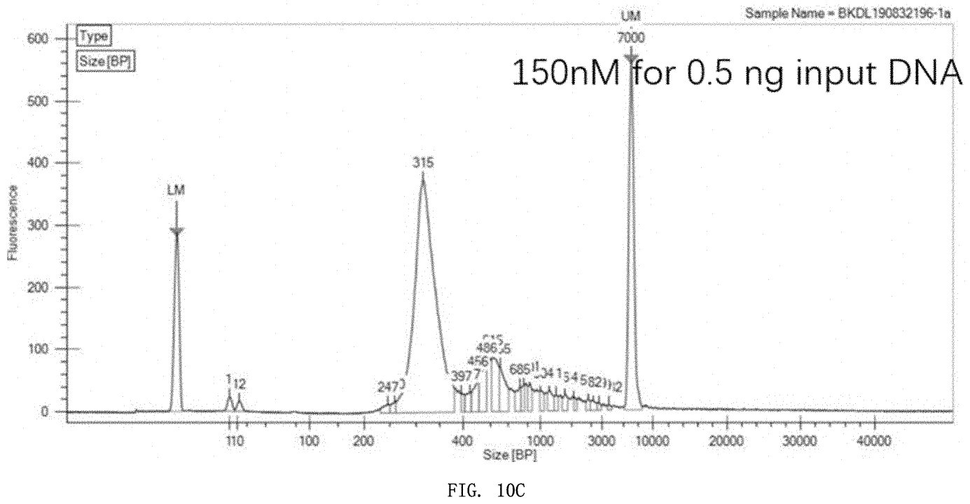

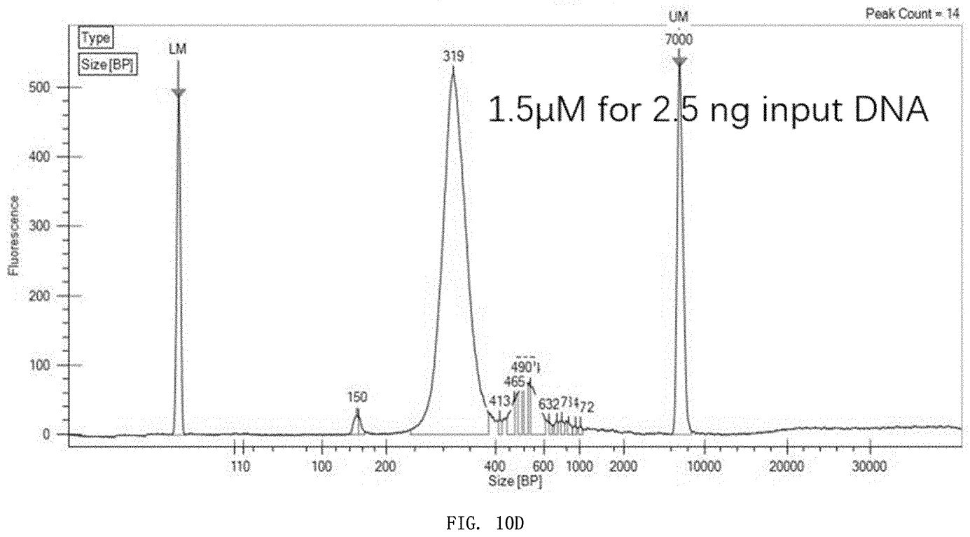

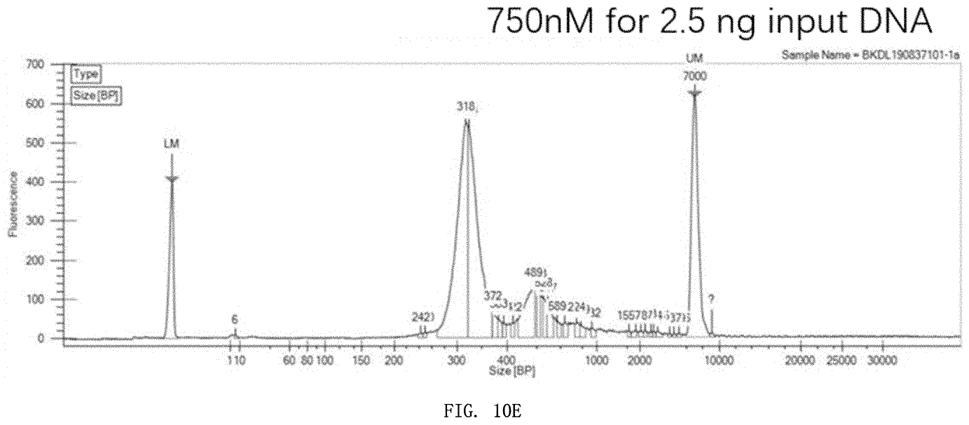

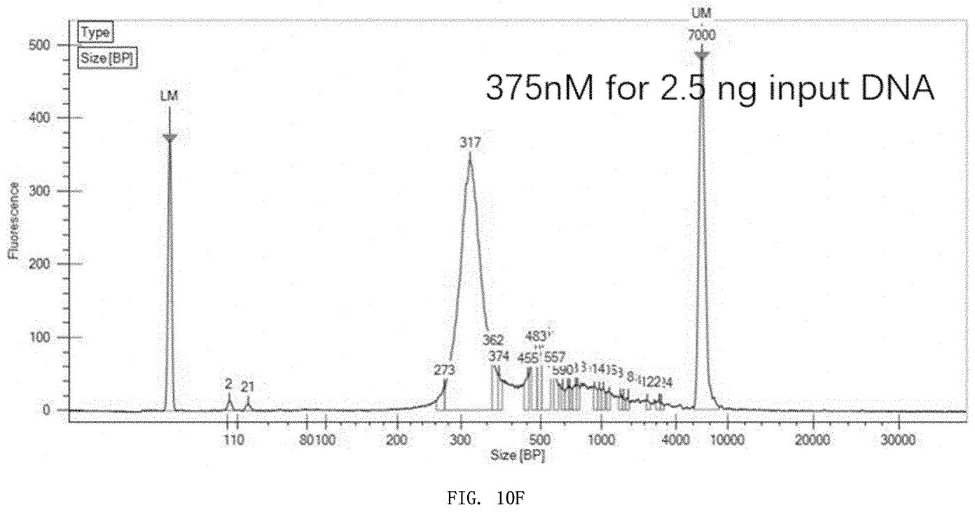

LabChip was used to detect the size of the purified fragments and analyze the residual adapter dimer in the library. It can be seen from FIGS. 10A-10F that there were obvious adapter dimer residues in the reaction system with 750 nM and 375 nM adapter for 0.5 ng input DNA, but not in the reaction system with 150 nM adapter; and there were obvious adapter dimer residues in the reaction system with 1.5 μM for 2.5 ng input DNA, but not in the reaction system with 750 nM and 375 nM adapter. However, the library products for the reaction system with 2.5 ng input DNA containing 375 nM adapter were significantly lower than that constructed for the reaction system containing 750 nM adapter. In conclusion, the optimal concentration of adapter for 0.5 ng input DNA is ≥150 nM, and the optimal concentration of adapter for 2.5 ng input DNA is ≥750 nM. The adapter was added in a volume of 5 μL to the reaction system.

The calculation of the molar ratio between the adapters and insert cfDNA fragments is:

adapter:insert molar ratio=(CᵢVᵢ):(Mⱼ/(2LⱼMWⱼ))

Cᵢ is the molarity of the adapter, Vᵢ is the volume of the adapter, Mⱼ is the mass of the input cfDNA, Lⱼ is the length of the input cfDNA, MWⱼ is the molecular weight of the dNTP. Because the cfDNA is double strand DNA, so it was multiplied by 2. Based on this formula, the adapter:insert ratio used in the method described herein has a molar ratio of about 100:1˜200:1 (e.g., about 162:1).

TABLE 14
Library construction parameter optimization

| | Input | | | Library |
|---|---|---|---|---|
| Sample | cfDNA | Adapter | PCR | concentration |
| No. | (ng) | concentration | cycles | (ng/ul) |
| S35 | 0.5 | 150 nM | 14 | 25 |
| | 0.5 | 150 nM | 13 | 11.5 |
| | 0.5 | 150 nM | 12 | 3.4 |
| | 0.5 | 150 nM | 11 | 1.08 |
| S36 | 2.5 | 750 nM | 12 | 38.2 |
| | 2.5 | 750 nM | 11 | 26.5 |
| | 2.5 | 750 nM | 10 | 7.6 |
| | 2.5 | 750 nM | 9 | 1.32 |

It can be seen from the above results, with the increase of PCR cycle number, library yield increases significantly. But overmuch PCR cycles will reduce the detection rate for original DNA, and it is not enough for quality requirements of sequencing by less PCR cycles (the sequencing service requires 50 ng for each library for sequencing two times. In case of insufficient data amount of the first sequencing, supplementary test is required. That is, library concentration should not be less than 1.43 ng/μL).

The number of PCR cycles can be determined using the formula:

MᵢCRᵢARᵢPRᵢ₁PRᵢ₂(2ⁿ−2n)=Mⱼ

wherein Mᵢ is the mass of input DNA, CRᵢ is the library conversion efficiency, ARᵢ is the PCR amplification rate, PRᵢ₁ is the purification rate of the ligation products, PRᵢ₂ is the purification rate of the PCR products, n is the PCR cycle, Mⱼ is the mass of the PCR product. Mⱼ should be more than 50 ng.

Due to the carrier RNA present in the eluted cfDNA sample, the mass of cfDNA in the sample is very low. Therefore, when the volume of the plasma is less than 1 mL in the steps of cfDNA extraction, Mᵢ should be multiplied by 0.2, which is the approximate fraction of cfDNA in the sample; when the volume of the plasma is about 1 mL to 2 mL in the steps of cfDNA extraction, Mᵢ should be multiplied by 0.6, which is the approximate fraction of cfDNA in the sample; when the volume of the plasma is about 2 mL to 3 mL in the steps of cfDNA extraction, Mᵢ should be multiplied by 0.8, which is the approximate fraction of cfDNA in the sample. The above formula can be applied directly when the volume of the plasma is more than 3 mL.

In the method described herein, the library conversion efficiency is about 60%, the PCR amplification rate is about 95%, the purification rate of the ligation products is about 80%, and the purification rate of the PCR products is about 80%.

TABLE 15
Index concentration adjustment

| | Index | PCR |
|---|---|---|
| Input cfDNA | concentration | cycle |
| 1 ng ≤ X ≤ 2.5 ng | 750 nM | 10 |
| 0.5 ng ≤ X < 1 ng | 150 nM | 12 |

### Example 8: Protein Tumor Marker Detection

This experiment was performed to evaluate the performance of the Roche Cobas E411 platform for detecting tumor protein markers at low plasma levels. The platform is an electrochemistry luminescence automatic immunoassay analyzer. It has certain requirements for sample volumes depending on the types and quantities of the project. The 7 protein tumor markers CEA, AFP, CA 125, CA 72-4, CA 19-9, CA 15-3, and CYFRA 21-1 were detected in the method described herein, and the minimum plasma volume was 300 μL. But capillary blood contained little plasma, and it was speculated that it could achieve the same detection effect by adding the sample diluent into the plasma. 8 samples were selected and 80 μL plasma of each sample was pipetted and 240 μL the dilution was added to achieve 4 times dilution. The sample was then loaded on the machine to detect the seven protein tumor markers.

The following is the test results of each sample:

TABLE 16
Tumor marker detection

| Sample | | | CA 19- | CA | CA 15- | CYFRA | CA 72- | |
|---|---|---|---|---|---|---|---|---|
| name | AFP < 11.3 | CEA < 4 | 9 < 27 | 125 < 35 | 3 < 25 | 21-1 < 3.3 | 4 < 6.9 | Note |
| S1 | 0.771 | 204.9 | 414.1 | 103.8 | 31.82 | 3.09 | 3.83 | The result of S1 |
| | <0.500 | 57.22 | 97.15 | 28.36 | 6.03 | 1.02 | 2.04 | The result of S1 |
| | | | | | | | | with dilution |
| | <2.0 | 228.88 | 388.6 | 113.44 | 24.12 | 4.08 | 8.16 | The result of S1 |
| | | | | | | | | with dilution |
| | | | | | | | | multiplies the |
| | | | | | | | | dilution ratio |
| S2 | 612.4 | 3.42 | 26.28 | 322.7 | 22.14 | 1.61 | 53.98 | The result of S2 |
| | 166.3 | 0.7 | 8.45 | 83.11 | 3.94 | 0.522 | 12.74 | The result of S2 |
| | | | | | | | | with dilution |
| | 665.2 | 2.8 | 33.8 | 332.44 | 15.76 | 2.088 | 50.96 | The result of S2 |
| | | | | | | | | with dilution |
| | | | | | | | | multiplies the |
| | | | | | | | | dilution ratio |
| S3 | 1.53 | 2.02 | 3.36 | 1311 | 75.48 | 15.33 | 102.3 | The result of S3 |
| | <0.500 | 0.29 | 3 | 373.4 | 19.62 | 4.88 | 23.18 | The result of S3 |
| | | | | | | | | with dilution |
| | <2.0 | 1.16 | 12 | 1493.6 | 78.48 | 19.52 | 92.72 | The result of S3 |
| | | | | | | | | with dilution |
| | | | | | | | | multiplies the |
| | | | | | | | | dilution ratio |
| S4 | 1.42 | 33.98 | 10.06 | 13.56 | 4.96 | 5.24 | 43.61 | The result of S4 |
| | <0.500 | 9.41 | 4.84 | 3.68 | <1.00 | 1.64 | 12.09 | The result of S4 |
| | | | | | | | | with dilution |
| | <2.0 | 37.64 | 19.36 | 14.72 | <4.00 | 6.56 | 48.36 | The result of S4 |
| | | | | | | | | with dilution |
| | | | | | | | | multiplies the |
| | | | | | | | | dilution ratio |
| S5 | 1.09 | 166.8 | 8.76 | 111.6 | 29.32 | 1.98 | 0.872 | The result of S5 |
| | <0.500 | 46.4 | 4.35 | 29.28 | 5.56 | 0.563 | 1.31 | The result of S5 |
| | | | | | | | | with dilution |
| | <2.0 | 185.6 | 17.4 | 117.12 | 22.24 | 2.252 | 5.24 | The result of S5 |
| | | | | | | | | with dilution |
| | | | | | | | | multiplies the |
| | | | | | | | | dilution ratio |
| S6 | 6.23 | 6.47 | 212 | 27.09 | 16.79 | 9.7 | 4.59 | The result of S6 |
| | 0.73 | 1.42 | 62.51 | 6.89 | 2.34 | 2.9 | 2.15 | The result of S6 |
| | | | | | | | | with dilution |
| | 2.92 | 5.68 | 250.04 | 27.56 | 9.36 | 11.6 | 8.6 | The result of S6 |
| | | | | | | | | with dilution |
| | | | | | | | | multiplies the |
| | | | | | | | | dilution ratio |
| S7 | 708.3 | 1.27 | 28.26 | 8.9 | 10.64 | 3.86 | 1.62 | The result of S7 |
| | 207.5 | <0.200 | 9.49 | 2.63 | 1.28 | 1.33 | 1.34 | The result of S7 |
| | | | | | | | | with dilution |
| | 830 | <0.800 | 37.96 | 10.52 | 5.12 | 5.32 | 5.36 | The result of S7 |
| | | | | | | | | with dilution |
| | | | | | | | | multiplies the |
| | | | | | | | | dilution ratio |
| S8 | 619.4 | 1.59 | 30.43 | 18.9 | 49.96 | 10.56 | 2.65 | The result of S8 |
| | 165.2 | <0.200 | 10.26 | 5.29 | 11.46 | 3.33 | 1.7 | The result of S8 |
| | | | | | | | | with dilution |
| | 660.8 | <0.800 | 41.04 | 21.16 | 45.84 | 13.32 | 6.8 | The result of S8 |
| | | | | | | | | with dilution |
| | | | | | | | | multiplies the |
| | | | | | | | | dilution ratio |

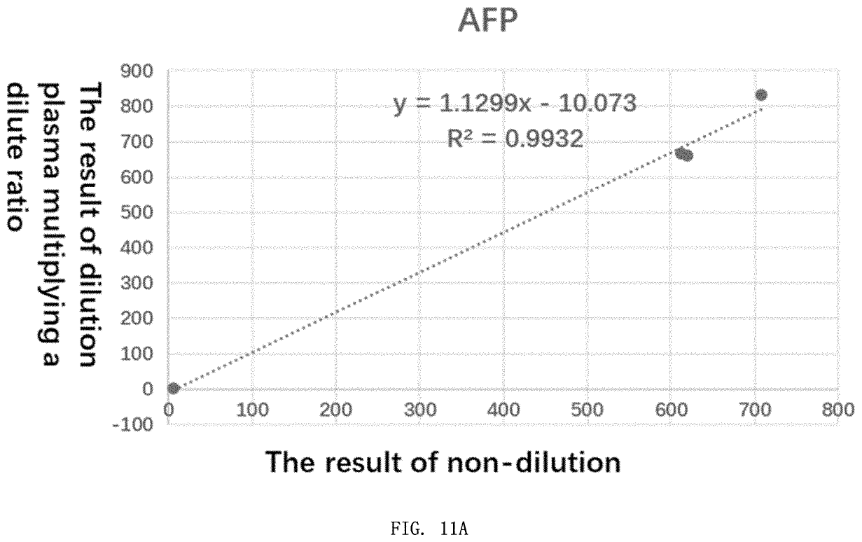

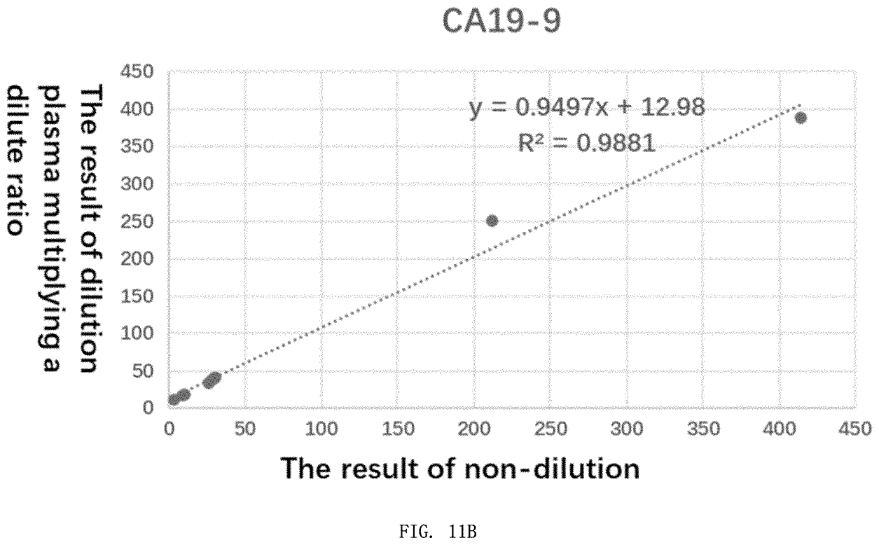

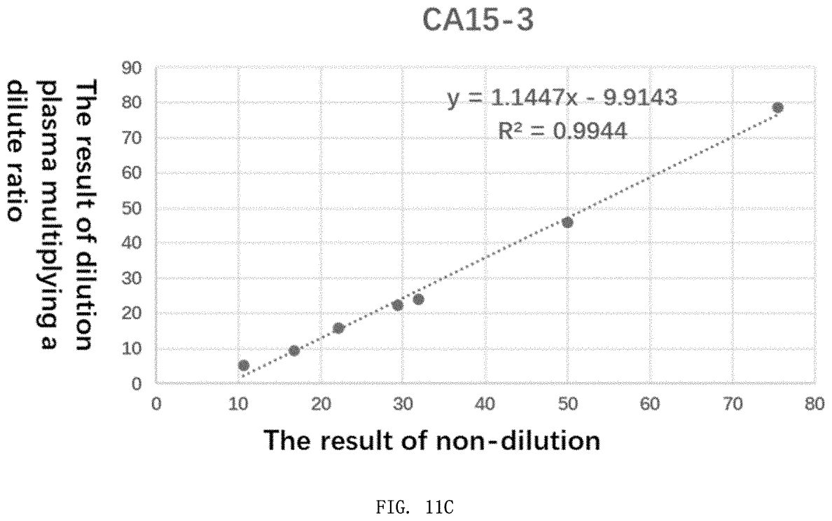

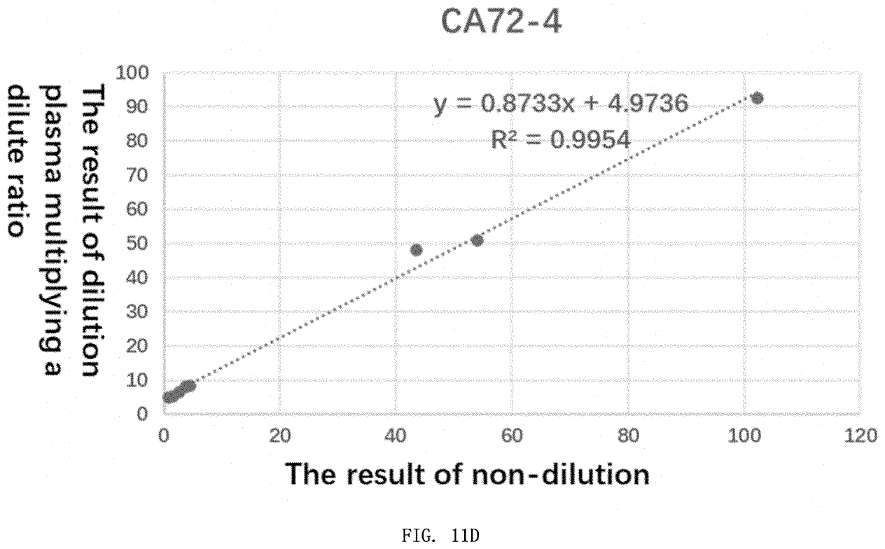

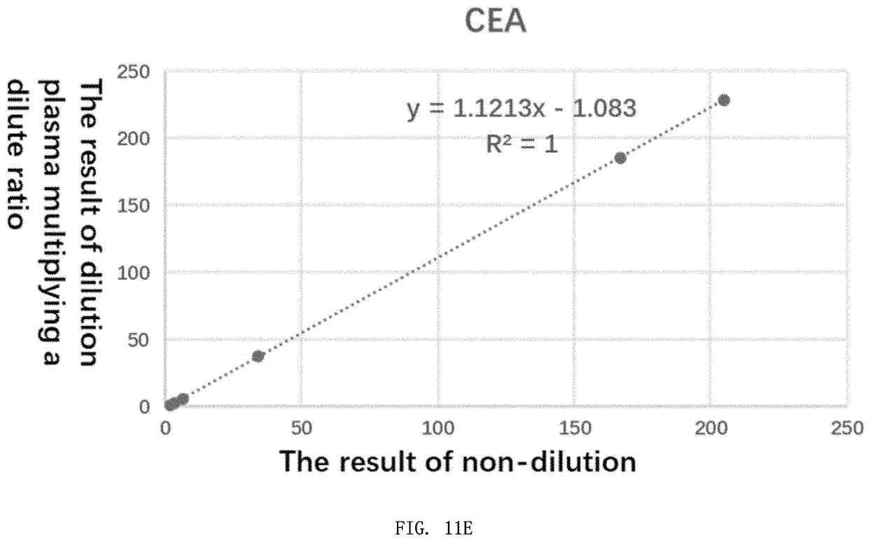

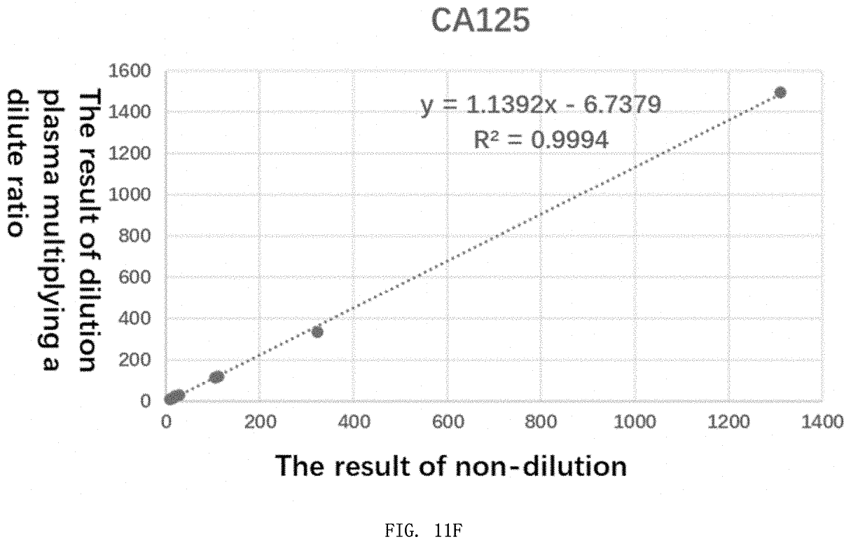

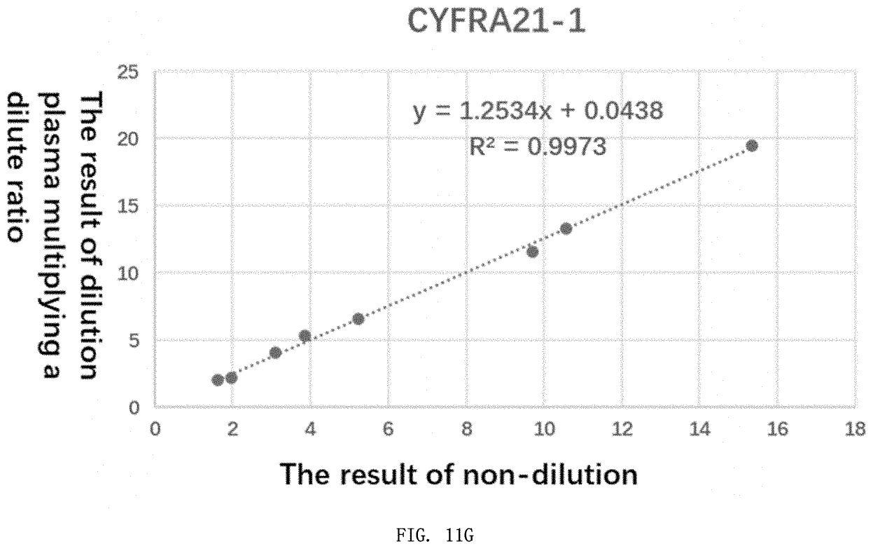

At the same time, we compared the result of each sample with dilution multiplies the dilution ratio with the result of the origin sample. The result is shown in FIGS. 11A-11G It can be seen that the correlation coefficient R² is more than 0.98. Therefore, it was found that the protein tumor markers can be effectively detected even when the plasma volume is reduced.

In conclusion, the present disclosure has established a method of cancer screening from capillary blood samples. This method uses BD Microtainer (a disposable capillary blood collection container) (Brand: BD, catalog number: 365974) and disposable blood collection device (Brand: BD, catalog number: 366594) to collect capillary blood. Carrier RNA and 2 elution operations were added in the cfDNA extraction process to increase cfDNA extraction efficiency. Through a series of tests, the appropriate adapter concentration and the PCR cycles were found to improve the library construction efficiency. At the same time, it was found through the test that the same detection outcome could be achieved when the amount of library construction reagent was reduced for little cfDNA and this reduced the cost. The use of sample diluents to dilute the samples enabled detection of protein tumor markers using the Roche Cobas E411 instrument even in very small volumes.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of isolating circulating free nucleic acid from a capillary blood plasma sample, the method comprising:
(1) diluting no more than 500 μL of capillary blood plasma sample to a final volume of about 1 mL using phosphate-buffered saline (PBS), thereby obtaining a diluted capillary blood plasma sample;
(2) lysing the diluted capillary blood plasma sample using protease K and buffer ACL, thereby obtaining a lysed capillary blood plasma sample;
(3) mixing the lysed capillary blood plasma sample with a binding buffer;
(4) loading the mixture in step (3) to a silica membrane column, to allow the binding of DNA to the silica membrane column;
(5) washing and drying the silica membrane column bound by DNA in step (4);
(6) eluting the DNA bound to the silica membrane column using 50-100 μL, preferably 50-60 μL of nuclease-free water (ddH₂O), thereby obtaining an eluted sample;
(7) loading the eluted sample to the silica membrane column, thereby obtaining the circulating free nucleic acid from the capillary blood sample.

2. The method of claim 1, wherein the lysing comprises:
(a) mixing the protease K, a carrier RNA, a lysis buffer, and the diluted capillary blood plasma sample,
(b) incubating the mixture of (a) for about 30 minutes at about 60° C.,
wherein the diluted capillary blood plasma sample has a volume of about 0.5-1.5 mL, the protease K has a volume of about 80-120 μL, the lysis buffer has a volume of about 750-850 μL, and the carrier RNA is used in an amount of about 0.5-1.5 μg.

3. The method of claim 1, wherein the mixture in step (3) is incubated on ice for about 5 minutes before step (4), and/or the drying of step (5) is performed in a metal bath under 56° C. for about 10 minutes.

4. The method of claim 1, wherein the method further comprises detecting and/or quantifying one or more protein tumor markers, wherein the protein tumor markers are selected from CEA, AFP, CA 125, CA 72-4, CA 19-9, CA 15-3, and CYFRA 21-1.

5. The method of claim 1, wherein the plasma sample is obtained using the following steps:
(i) collecting no more than 500 μL of capillary blood using a tube containing K2-EDTA anticoagulant;
(ii) performing centrifugation on the capillary blood under 1600 g and 4° C. for about 10 minutes, collecting the supernatant and removing the blood cells;
(iii) performing centrifugation on the supernatant obtained in step (ii) under 16000 g for about 10 minutes, collecting the supernatant and removing impurities including cell debris, thereby obtaining the plasma.

6. A method for preparing a circulating free nucleic acid sequencing library from a capillary blood sample, the method comprising:
(a) end repairing and A-tailing the circulating free nucleic acid of claim 1, thereby obtaining A-tailed circulating free nucleic acid, wherein the starting amount of the circulating free nucleic acid is about 0.5-2.5 ng;
(b) ligating an adapter to the A-tail of the circulating nucleic acid, thereby obtaining an adapter-ligated library of circulating free nucleic acid;
(c) amplifying the adapter-tagged nucleic acid in the library.

7. The method of claim 6, wherein
in step (b), the concentration of adapter can be determined using the formula:
(CᵢVᵢ):(Mⱼ/(2LⱼMWⱼ))=about 100:1˜200:1 (e.g., about 162:1),
wherein Cᵢ is the molarity of the adapter, Vᵢ is the volume of the adapter, Mⱼ is the mass of the input cfDNA, Lⱼ is the length of the input cfDNA, and MWⱼ is the molecular weight of the dNTP;
and/or in step (c), the number of PCR cycles can be determined using the formula:
MᵢCRᵢARᵢPRᵢ₁PRᵢ₂(2ⁿ−2n)=Mⱼ
wherein Mᵢ is the mass of input DNA, CRᵢ is the library conversion efficiency, ARᵢ is the PCR amplification rate, PRᵢ₁ is the purification rate of the ligation products, PRᵢ₂ is the purification rate of the PCR products, n is the PCR cycle, and Mⱼ is the mass of the PCR product.

8. The method of claim 6, wherein in step (b), when the starting amount of the circulating free nucleic acid is not less than 0.5 ng and less than 1 ng, the concentration of the adapter used in the ligation is about 100-200 nM; when the starting amount of the circulating free nucleic acid is not less than 1 ng and less than 2.5 ng, the concentration of the adapter used in the ligation is 700-800 nM; and/or
wherein in step (c), when the starting amount of the circulating free nucleic acid is not less than 0.5 ng and less than 1 ng, the amplification is performed for 10-15 rounds; when the starting amount of the circulating free nucleic acid is not less than 1 ng and less than 2.5 ng, the amplification is performed for 8-12 rounds.

9. A method of sequencing circulating free nucleic acid in a capillary blood sample, the method comprising:
preparing a sequencing library using the method of claim 6; and sequencing the library thereby obtaining sequencing data.

10. The method of claim 9, the method further comprises:
analyzing the sequencing data to obtain information on genetic copy number variation and fragmentation pattern of the nucleic acid.

11. A method of detecting or assessing the risk of cancer in a subject, the method comprising:
(1) obtaining no more than 500 μL of a capillary blood plasma sample in the subject;
(2) diluting no more than 100 μL of plasma to obtain a diluted plasma sample, wherein the plasma is diluted about 1 to about 5 folds (preferably 4 folds);
(3) analyzing the diluted plasma sample to detect and/or quantify one or more protein tumor markers; wherein the protein tumor markers are one or more of CEA, AFP, CA 125, CA 72-4, CA 19-9, CA 15-3, and CYFRA 21-1;
(4) sequencing and analyzing circulating free nucleic acid in the remaining plasma sample to obtain information on gene copy number variation and fragmentation pattern; and
(5) detecting and/or assessing the risk of cancer in the subject based on the detection of the protein tumor markers, information on gene copy number variation and the fragmentation pattern.

12. A device for sequencing the circulating free nucleic acid from capillary blood sample, the device comprising:
a device for preparing a sequencing library for carrying out the method of claim 6; and
a sequencing device for sequencing the library to obtain sequencing data.

13. The device of claim 12, further comprising:
a device for analyzing the sequencing data to obtain information on gene copy number variation and fragmentation pattern.

14. A system for determining the probability of having cancer in a subject, the system comprising:
a device to obtain no more than 500 μL of capillary blood plasma sample;
a protein analyzing device, the device is designed to:
dilute no more than 100 μL, preferably 80 μL of the plasma sample, thereby obtaining a diluted plasma sample, wherein the sample is diluted 1-5 times; and
detect protein tumor markers in the diluted plasma sample, wherein the protein tumor markers are one or more of CEA, AFP, CA 125, CA 72-4, CA 19-9, CA 15-3, and CYFRA 21-1;
a device for sequencing and analyzing circulating free nucleic acid in the rest of the capillary blood plasma sample, to obtain information on gene copy number variation and fragmentation pattern;
a device for analyzing the results of the detection of the protein tumor markers, gene copy number variation and fragmentation pattern, thereby obtaining the probability of having cancer in a subject.

15. A sequencing library, prepared by the method of claim 6.

16. The method of claim 1, wherein the circulating free nucleic acid is circulating free DNA (cfDNA).

17. The method of claim 1, wherein the capillary blood plasma sample is obtained from a subject having cancer, and cfDNA isolated from every 100 μL of blood sample is about 0.5-100 ng.

18. The method of claim 6, further comprising one or more purifying step(s) of the ligated library of circulating free nucleic acid to remove excess adapters.

19. The method claim 18, wherein the purifying step(s) is performed using magnetic beads.

20. The method claim 19, wherein the ratio of the volume of the magnetic beads and the volume of the ligated library of circulating free nucleic acid is about 0.8:1.
